SEQUENCE LISTING (31 sequences)

SEQ ID NO: 1 - DNA, 204 bp, artificial
  complete CDS of codon-optimised CspB gene
atgctagagggcaaagtgaagtggttcaacagcgagaagggattcggctttatcgaagtggaaggccaggatgacgtgtttgttcacttctctgccatccaaggggaaggattcaagacactggaggaaggacaggcagtctcattcgagatagtcgaggggaatagaggacctcaagctgcgaacgttaccaaagaggcttga

SEQ ID NO: 2 - protein, 67 aa, Bacillus subtilis
MLEGKVKWFNSEKGFGFIEVEGQDDVFVHFSAIQGEGFKTLEEGQAVSFEIVEGNRGPQAANVTKEA

SEQ ID NO: 3 - DNA, 213 bp, artificial
  complete cds of E. coli cspA gene, optimised
atgtccggtaaaatgactggtatcgtaaaatggttcaacgctgacaaaggcttcggcttcatcactcctgacgatggctctaaagatgtgttcgtacacttctctgctatccagaacgatggttacaaatctctggacgaaggtcagaaagtgtccttcaccatcgaaagcggcgctaaaggcccggcagctggtaacgtaaccagcctgtaa

SEQ ID NO: 4 - protein, 70 aa, Escherichia coli
MSGKMTGIVKWFNADKGFGFITPDDGSKDVFVHFSAIQNDGYKSLDEGQKVSFTIESGAKGPAAGNVTSL

SEQ ID NO: 5 - DNA, 423 bp, Arabidopsis thaliana
atggcggatacgccttcgagcccagctggagatggcggagaaagcggcggttccgttagggagcaggatcgataccttcctatagctaatatcagcaggatcatgaagaaagcgttgcctcctaatggtaagattggaaaagatgctaaggatacagttcaggaatgcgtctctgagttcatcagcttcatcactagcgaggccagtgataagtgtcaaaaagagaaaaggaaaactgtgaatggtgatgatttgttgtgggcaatggcaacattaggatttgaggattacctggaacctctaaagatatacctagcgaggtacagggagttggagggtgataataagggatcaggaaagagtggagatggatcaaatagagatgctggtggcggtgtttctggtgaagaaatgccgagctgg

SEQ ID NO: 6 - protein, 141 aa, Arabidopsis thaliana
MADTPSSPAGDGGESGGSVREQDRYLPIANISRIMKKALPPNGKIGKDAKDTVQECVSEFISFITSEASDKCQKEKRKTVNGDDLLWAMATLGFEDYLEPLKIYLARYRELEGDNKGSGKSGDGSNRDAGGGVSGEEMPSW

SEQ ID NO: 7 - DNA, 1323 bp, artificial
  Complete CDS of Brassica napus cultivar Hua shuang No. 5 transcription factor LAS, splice sites removed
atgcttacttccttcaaatcctctagctcctcctccgaagatgccaccgagaatcctcctcctcctcctccgttatgcctcgcctcatcttctgccgcaacatccgccgctcatcacctccgtcgtctactattcacagctgcggatttcatctctcagtccaacgtctccgccgctcaaaacatactctcaatcctctcctcaaactcttccccttacggggactccacggagcggctcgtccatctcttcaccaaagccttgtccgtacggatcggcttgtctgaaaacactgccacgtggacagcgaacgaaatggcttctagctccacggtttttacaagcagtgtatgcaaagaacagttcttgtttcgaaccaagaacaacaacaactctgatctcgagtcttgttactatctttggctgaaccaactaacaccgtttattcggttctcccatttaacggcgaaccaagcgatcctcgacgcgactgagacaaacaacggtaacggagctttacatatacttgacttagatatatcacaaggacttcaatggcctccgttgatgcaagccctagccgagagatcatcatcaaaccctagcagtactccacctccttccctccgcataaccggatgtggtcgagatgtaaccgtattaaaccgaaccggagatcggttaacccggtttgctaactctctaggtcttcagtttcagtttcacacgcttgtgatcgctgaagaagacctcgccggacttttgcttcagatcagattattagctctctccgccgtacaaggagagtccatcgccgtcaactgcgtccacttccttcacagattctttaacgacgacggagacatgatcggtcacttcctgtcggcgatcaagagcttaaaccctagaatcgtgacaatggcggagagagaagcgaaccatggagatccttcgttcttgactagattctcagaggctttagatcatttcatggcgatatttgattcgttggaagcgactttgccgccaaacagcaaagagaggctaaccctagagcaacggtggttcggtatggagattttggatgttgtggcggcggaagcggcggagagaaagcaaagacatcggaggtttgaggtttgggaggagatgatgaagagacatggctttgctaacgtgccaataggaagctttgctttctctcaagctaagcttctgcttagactccattatccttcagaaggttataatcttcagtttctcaacgactctttgtttcttggatggaaaaatcgtcttctcttctccgtttcgtcgtggaaa

SEQ ID NO: 8 - protein, 441 aa, brassica napus
MLTSFKSSSSSSEDATENPPPPPPLCLASSSAATSAAHHLRRLLFTAADFISQSNVSAAQNILSILSSNSSPYGDSTERLVHLFTKALSVRIGLSENTATWTANEMASSSTVFTSSVCKEQFLFRTKNNNNSDLESCYYLWLNQLTPFIRFSHLTANQAILDATETNNGNGALHILDLDISQGLQWPPLMQALAERSSSNPSSTPPPSLRITGCGRDVTVLNRTGDRLTRFANSLGLQFQFHTLVIAEEDLAGLLLQIRLLALSAVQGESIAVNCVHFLHRFFNDDGDMIGHFLSAIKSLNPRIVTMAEREANHGDPSFLTRFSEALDHFMAIFDSLEATLPPNSKERLTLEQRWFGMEILDVVAAEAAERKQRHRRFEVWEEMMKRHGFANVPIGSFAFSQAKLLLRLHYPSEGYNLQFLNDSLFLGWKNRLLFSVSSWK

SEQ ID NO: 9 - DNA, 1029 bp, artificial
  complete CDS of GmRD22, C843G silent mutation
atggagtatcgtctcctacccatttttactttactcaatcttgcactggtggcaatccatgctgctttacctcctgaagtttactggaagtcggtgcttcctactacgccaatgccaaaagccatcactgatatcctttaccccgattgggtggaagagaaaagtacctcagtgaatgttggaggcaagggcgtaaacgtgcatgcaggaaaaggaggaggtggcaccaatgtcaacgttggtggaaaaggatcaggcggaggcgtgaacgtgcatgcaggtcacaagggaaagccagtgcatgtttctgttggctcaaagtctccattcaattacatctacgcttcaacggagactcaattacacgatgaccccaacgtcgcactcttcttcttggaaaaggacttgcatcccggaacaaagttgaacttgcacttcaccaccagttccaatattcaagccacattcttgccacgccaagttgcggattctatacccttttcatccagcaaggtggaggttgtattcaacaagttttccgtaaaacccgggtcagaggaggcccagatcatgaagaatactctcagtgagtgtgaagagggtggcatcaaaggagaggaaaagtactgtgccacttcgcttgaatccatgattgatttcagcacttccaagcttggaaaaaatgttgaggttgtgtccacggaagtagtggaggacaaggaaacgggattgcagaaatacaccgtagcaccgggagtgaacaagttatcaggggacaaggctgttgtgtgccacaagcagaactacccttatgctgttttttactgtcacaaaactgagacgacaagagcttactctgtgcctttggagggtgctaatggggttagggttaaagcggtagcagtgtgccacactcacacgtcggaatggaaccctaaacatttggcctttcaagtgctcaaagttaagccaggaaccgttcctgtctgccacttcctacctgaggatcatgttgtttgggttcccaag

SEQ ID NO: 10 - protein, 343 aa, soybean
MEYRLLPIFTLLNLALVAIHAALPPEVYWKSVLPTTPMPKAITDILYPDWVEEKSTSVNVGGKGVNVHAGKGGGGTNVNVGGKGSGGGVNVHAGHKGKPVHVSVGSKSPFNYIYASTETQLHDDPNVALFFLEKDLHPGTKLNLHFTTSSNIQATFLPRQVADSIPFSSSKVEVVFNKFSVKPGSEEAQIMKNTLSECEEGGIKGEEKYCATSLESMIDFSTSKLGKNVEVVSTEVVEDKETGLQKYTVAPGVNKLSGDKAVVCHKQNYPYAVFYCHKTETTRAYSVPLEGANGVRVKAVAVCHTHTSEWNPKHLAFQVLKVKPGTVPVCHFLPEDHVVWVPK

SEQ ID NO: 11 - DNA, 855 bp, Brassica rapa
atggctatggaagagaaactcgtgatcaacgaactggaacaagggagagagcttgcccaacgtttgatgagcaatctcaaagacacttcctcaatcgaatccagcaagaacttgatctctgagatcctcagtatctaccagaatgctatttccatgttagacgacaagaaggtccttaaacgtagccgtgagatcgatgacaaagattctaagaacgtgataaaaaagaggcaagtgtttgagaagaagacagagaaagttagtttctttgtcggagcaggacaagaaaagggttccattgatgatggttattgctggagaaagtacggtcaaaaagagattcatggatccattaatccaagaggatatttcagatgcacgcatcgattcacacagaactgtttagcagtgaagcaagtccaaaaatcagacagagatccttccattttcgaagtgaagtatgtcgggagccacacttgtaacaacactactacgtccccaaagacaccgaacttctctatttcgatgttccaacaagaagacatcaaaccgacgaaaacagaggaagcgatgatgagtcttgaagatctcgagagcactaagaacattttcagaacgttttctttctccaactacgagattgagaatgctggtggtggttggaaaggcaacctcttccatgaggatcagctgtctcctgctgcgactacgtcagggtctggaatcaccagcgaggttgcaacagctcctgcttccgttgagaactcggagactgcagattcgtatttctcgtctttggacaatattatcgactttggaccggattggttgctgtcgtgtgacgttttgaattgg

SEQ ID NO: 12 - protein, 285 aa, brassica rapa
MAMEEKLVINELEQGRELAQRLMSNLKDTSSIESSKNLISEILSIYQNAISMLDDKKVLKRSREIDDKDSKNVIKKRQVFEKKTEKVSFFVGAGQEKGSIDDGYCWRKYGQKEIHGSINPRGYFRCTHRFTQNCLAVKQVQKSDRDPSIFEVKYVGSHTCNNTTTSPKTPNFSISMFQQEDIKPTKTEEAMMSLEDLESTKNIFRTFSFSNYEIENAGGGWKGNLFHEDQLSPAATTSGSGITSEVATAPASVENSETADSYFSSLDNIIDFGPDWLLSCDVLNW

SEQ ID NO: 13 - DNA, 939 bp, artificial
  complete HaHB1-Helianthus annuus HD-Zip subfamily I trancription factor; splice sites removed G561A, G750A
atgacttgcactggaatggctttcttctcctccaatttcatgttacaatcctcccaagaagatgaccatcatgcccctacatctctctctccaatcctcccaccttgcagtaccaccactcaagatttcagtggtgctgctttcttgggaaaaagatctatgtcttcttactcaggtttgaacaacaacaacatggatggatgtgatcaagaagggaacatgaatggagaagatgagttatcagatgatggatcacagcttcttgcaggagagaaaaagaggagattaaacatggaacaagtgaagacacttgagagaaactttgagttaggaaataagcttgaacctgagaggaaaatgcaacttgcaagagcacttggactacaaccaagacagattgctatatggtttcaaaacagaagagctagatggaaaactaaacagttggaaaaagactatgatgccctcaagagacagtttgaagctgttaaagctgagaatgattcactccaatctcaaaatcataaacttcatgctgagataatggcactaaaaaatagagagccagcagaactaatcaacctcaacataaaagaaacagaaggatcttgcagcaaccgaagcgaaaacagctctgaaatcaaactagacatctcaagaacaccggctaccgatagccctttatcatcacaccatcaacaccaacaccagccaatacctaatctttttccatcgtcgaatatcgatagacctaattcgaataacattgtggcgcatcaacttttccacaattcgtcatcaaggccggcagatcatcaacttcattgccacaaactcgatcaatcgaatgccattaaagaagaatgttttagcacaatgtttgttggtatggatgatcaatcagggttttggccatggttggaacaaccacaattcaat

SEQ ID NO: 14 - protein, 313 aa, artificial
  HaHB1-Helianthus annuus HD-Zip subfamily I trancription factor; splice sites removed G561A, G750A
MTCTGMAFFSSNFMLQSSQEDDHHAPTSLSPILPPCSTTTQDFSGAAFLGKRSMSSYSGLNNNNMDGCDQEGNMNGEDELSDDGSQLLAGEKKRRLNMEQVKTLERNFELGNKLEPERKMQLARALGLQPRQIAIWFQNRRARWKTKQLEKDYDALKRQFEAVKAENDSLQSQNHKLHAEIMALKNREPAELINLNIKETEGSCSNRSENSSEIKLDISRTPATDSPLSSHHQHQHQPIPNLFPSSNIDRPNSNNIVAHQLFHNSSSRPADHQLHCHKLDQSNAIKEECFSTMFVGMDDQSGFWPWLEQPQFN

SEQ ID NO: 15 - DNA, 1734 bp, Nicotiana tabacum
atggcttccttaggcttcttgttcttcttcttgttgccattgattttgcttgagttatcttcatctaggtcagtaatggcagcaaaaacacggcattttaaatgggacgtggaatatattcattggtcaccagatggtgaagaaagtgtagtaatgggaatcaatggacagtttcctggtccaactattagggcaaaagctggtgatactgttgctgttcatcttactaacaagctacatactgaaggtgttgtcattcattggcatggaatccgacagatcggaacaccatgggctgatggaactgcagcaatttcccaatgcgccattaaccctggagagacatttctctataggtttaaagttgataaggcagggacatacttctaccatggacactatggaatgcaaagatcagcagggctatatggttcactaatagtggaagttggagaaggtgaaaaagaaccattccattatgatggagaattcaatttattgcttagtgactggtggcacaaaggttcccatgaacaagaagttgacctctcttccaatcctcttcgttggattggtgaaccccagacattgttgctaaatgggagaggtcaatacaattgttcacttgcggcgcggtttagcaaaccaccacttccacagtgcaagttaagagggggtgaacagtacgcaccccagattctgcgcgtgcgtcccaacaagatttacaggcttagggtggcaagtactactgcattgggttcactcagcttggccattgggggtcacaagatggtggtagtagaagcagatggaaactatgttcaaccattttcagtacaagacatggacatttattcaggtgaaagctattcagtccttttcaaaacagatcaagatcctaccaaaaactattggatttcaataaatgtaagaggaagagaaccaaaaacacctcaaggcctcaccttattaaactatcttccaaattctgcatccaaatttccaactttaccaccacctatagcacccctttggaatgattataaccatagtaagtcattttctaacaaaatttttgccttaatgggatcacctaagccaccacctcagaaccatcgtcgtatcatcctgctcaatactcagaacaaaatcgatggttacacgaaatgggctataaataacgtgtcgttggtcttgccaacgcaactttatttaggctcgattagatatggcataaacgcgtttgacacgaaacctccaccggacaacttccctaaggactatgatgtcctaaaacaagcaccaaattctaattctacatatggtaatggtgtgtatatgctaaagttcaatactacaattgacattatcctacaaaatgcaaatgccttagctaaagatgttagtgaaattcatccttggcatttgcatggacatgatttttgggtattgggatatggagaagggaaatttagtgaaaaagatgtcaagaagttcaatttaaagaatccaccattgagaaatactgctgtgatttttccctttggttggactgcactaagatttgtgacagataatcctggagtttgggcttttcattgtcatattgagccacatttacatatgggaatgggagttatatttgctgaaggtgttcatcttgtcaagaaaatacctaaagaagctttggcttgtggtttgacagggaaaatgttgatgagtaacaagcataat

SEQ ID NO: 16 - protein, 578 aa, Nicotiana tabacum
MASLGFLFFFLLPLILLELSSSRSVMAAKTRHFKWDVEYIHWSPDGEESVVMGINGQFPGPTIRAKAGDTVAVHLTNKLHTEGVVIHWHGIRQIGTPWADGTAAISQCAINPGETFLYRFKVDKAGTYFYHGHYGMQRSAGLYGSLIVEVGEGEKEPFHYDGEFNLLLSDWWHKGSHEQEVDLSSNPLRWIGEPQTLLLNGRGQYNCSLAARFSKPPLPQCKLRGGEQYAPQILRVRPNKIYRLRVASTTALGSLSLAIGGHKMVVVEADGNYVQPFSVQDMDIYSGESYSVLFKTDQDPTKNYWISINVRGREPKTPQGLTLLNYLPNSASKFPTLPPPIAPLWNDYNHSKSFSNKIFALMGSPKPPPQNHRRIILLNTQNKIDGYTKWAINNVSLVLPTQLYLGSIRYGINAFDTKPPPDNFPKDYDVLKQAPNSNSTYGNGVYMLKFNTTIDIILQNANALAKDVSEIHPWHLHGHDFWVLGYGEGKFSEKDVKKFNLKNPPLRNTAVIFPFGWTALRFVTDNPGVWAFHCHIEPHLHMGMGVIFAEGVHLVKKIPKEALACGLTGKMLMSNKHN

SEQ ID NO: 17 - DNA, 2310 bp, artificial
  CDS of AVP1_ARATH with E229D mutation; splices sites removed A261G, C2004G, A2190T
atggtggcgcctgctttgttaccggagctctggacggagatccttgtaccgatttgtgcggtgattggtatcgccttttcgcttttccaatggtacgttgtatctcgcgtgaaactcacctctgacctcggcgcatcgtcttccggtggagctaacaatgggaagaatggatacggtgattatctaatcgaggaagaggaaggtgttaatgaccagagtgttgtcgctaagtgcgctgagattcagactgctatttccgagggtgcaacttcattcctattcacggagtacaaatatgttggtgtcttcatgattttctttgctgctgttatctttgttttcctcggctctgttgagggattcagcactgataacaagccttgtacttacgacaccaccagaacctgcaagcctgcattggctactgcagctttcagtaccattgctttcgtgcttggtgctgttacctctgttctatctggtttccttgggatgaagattgctacatacgctaatgctaggaccactttggaggcgaggaaaggtgttggaaaggcgttcattgttgcattcaggtctggtgctgtgatgggtttccttcttgcagcgagtggtctattggtgctttacattactatcaatgtgttcaagatctattacggagatgactgggaaggtctttttgacgctattactggttatggtcttggtgggtcttccatggctctctttggccgtgttggtggtgggatctacactaaggctgctgatgtcggcgctgaccttgtcggtaaaattgagaggaatattccagaggatgatccaagaaacccagctgtcattgctgataatgtcggtgacaatgttggtgacattgctggtatgggatctgatctctttggatcatatgctgaagcatcatgcgctgctcttgttgttgcctcgatctcatctttcggaatcaaccacgacttcactgccatgtgctacccattgctcatcagttcaatgggaatcttggtttgtttgatcacaactctctttgccactgacttctttgagattaagcttgtcaaggagattgaaccagcattgaagaaccagctcattatctcaactgttattatgactgttggtattgctattgtgtcatgggttggcttaccgacctcctttaccatcttcaactttggaacacaaaaagttgtcaagaactggcagctattcctttgtgtttgtgttggtctttgggctggactcattattggtttcgtcactgagtactacactagtaacgcctacagccctgtgcaagatgttgcagattcatgcagaactggtgcagctaccaatgttatcttcggccttgctcttggttacaaatccgtcattattccaatctttgctattgctatcagtatattcgttagcttcagctttgctgctatgtatggtgttgctgttgctgctcttggtatgctcagtaccattgccactggtttggcaattgatgcttatggtcccatcagtgacaatgctggtggtattgctgaaatggctggaatgagccaccgcatccgtgaaagaactgatgctcttgatgccgctggaaacaccactgctgctattggaaagggatttgccattggctctgctgccctagtctccttggctctctttggtgcctttgtgagccgtgcagggatccacaccgtagatgttttgacccctaaagttatcattgggctccttgttggtgccatgcttccttactggttctctgccatgacaatgaagagtgtgggaagtgcagctcttaagatggttgaagaagttcgcaggcagttcaacaccatccctggacttatggaaggaaccgcaaaaccagactacgccacatgtgtcaagatctccaccgatgcttccatcaaggaaatgatacctcctggttgccttgtcatgctcacacctctcattgttggtttcttctttggagttgagacgctctctggtgtcctcgccggatctcttgtatccggtgttcagatcgccatatcagcatctaacactggtggtgcctgggacaacgccaagaaatacatcgaggctggtgtatcagagcacgcaaagagccttggaccaaagggttcagagccacacaaggcagctgtgattggagacacaattggtgacccattgaaggatacttcaggaccttcattgaacatcctcatcaagctcatggctgttgagtctcttgtctttgctcccttcttcgccactcacggtggtatccttttcaagtacttc

SEQ ID NO: 18 - protein, 770 aa, artificial
  AVP1_ARATH of A. thaliana, with E229D mutation; splices sites removed A261G, C2004G, A2190T
MVAPALLPELWTEILVPICAVIGIAFSLFQWYVVSRVKLTSDLGASSSGGANNGKNGYGDYLIEEEEGVNDQSVVAKCAEIQTAISEGATSFLFTEYKYVGVFMIFFAAVIFVFLGSVEGFSTDNKPCTYDTTRTCKPALATAAFSTIAFVLGAVTSVLSGFLGMKIATYANARTTLEARKGVGKAFIVAFRSGAVMGFLLAASGLLVLYITINVFKIYYGDDWEGLFDAITGYGLGGSSMALFGRVGGGIYTKAADVGADLVGKIERNIPEDDPRNPAVIADNVGDNVGDIAGMGSDLFGSYAEASCAALVVASISSFGINHDFTAMCYPLLISSMGILVCLITTLFATDFFEIKLVKEIEPALKNQLIISTVIMTVGIAIVSWVGLPTSFTIFNFGTQKVVKNWQLFLCVCVGLWAGLIIGFVTEYYTSNAYSPVQDVADSCRTGAATNVIFGLALGYKSVIIPIFAIAISIFVSFSFAAMYGVAVAALGMLSTIATGLAIDAYGPISDNAGGIAEMAGMSHRIRERTDALDAAGNTTAAIGKGFAIGSAALVSLALFGAFVSRAGIHTVDVLTPKVIIGLLVGAMLPYWFSAMTMKSVGSAALKMVEEVRRQFNTIPGLMEGTAKPDYATCVKISTDASIKEMIPPGCLVMLTPLIVGFFFGVETLSGVLAGSLVSGVQIAISASNTGGAWDNAKKYIEAGVSEHAKSLGPKGSEPHKAAVIGDTIGDPLKDTSGPSLNILIKLMAVESLVFAPFFATHGGILFKYF

SEQ ID NO: 19 - DNA, 945 bp, artificial
  SNAC1 - Oryza sativa, Complete CDS, codon optimized
atgggaatgagacgagaacgagacgcagaagccgagcttaatctgccacctggctttcgttttcatccgactgatgatgaactagtggaacactacctttgcagaaaagcagctggtcaaaggctcccagttcctatcatagctgaggttgatctctataaatttgacccctgggatcttccggaaagggcattgtttggggctcgtgaatggtactttttcaccccacgcgataggaagtacccaaacggatctcgtccaaacagggccgctggaaacggttattggaaagcgactggagctgacaagccagtagctcctagggggagaacgctcggtattaaaaaggctctagttttctacgctggaaaagccccaagaggcgttaagactgactggattatgcatgaatatagactggctgatgctggaagagctgctgcgggggctaagaaaggtagtttgagattagacgattgggtcctctgtcggctatataacaagaagaacgagtgggagaagatgcaacaggggaaagaggttaaggaggaagcctcggatatggtgacttcccagtcgcatagccacacccactcttggggagaaacccgcacaccagagtctgagatcgttgataacgacccctttccggaattggattctttccctgcctttcaacctgcacctccacctgcaactgcaatgatggtcccgaagaaagaatcaatggacgacgcaacagcagcagctgctgctgccgcgacaatccctaggaataattcaagccttttcgtagatctttcctacgacgatattcaggggatgtatagcggcttagacatgttgcctcccggcgacgacttctattcctcattgttcgcaagtccccgtgtcaaaggaacaacacctcgagcaggtgctggaatgggaatggtgcctttttaa

SEQ ID NO: 20 - protein, 314 aa, Oryza sativa
MGMRRERDAEAELNLPPGFRFHPTDDELVEHYLCRKAAGQRLPVPIIAEVDLYKFDPWDLPERALFGAREWYFFTPRDRKYPNGSRPNRAAGNGYWKATGADKPVAPRGRTLGIKKALVFYAGKAPRGVKTDWIMHEYRLADAGRAAAGAKKGSLRLDDWVLCRLYNKKNEWEKMQQGKEVKEEASDMVTSQSHSHTHSWGETRTPESEIVDNDPFPELDSFPAFQPAPPPATAMMVPKKESMDDATAAAAAAATIPRNNSSLFVDLSYDDIQGMYSGLDMLPPGDDFYSSLFASPRVKGTTPRAGAGMGMVPF

SEQ ID NO: 21 - DNA, 720 bp, artificial
  IPT isopentenyl transferase IPT (E88G)
atggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcagacgaggagaccttcatgaacgtggccaaggccagagttaagcagatgttacgccctgctgcaggcctttctattatccaagagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtat

SEQ ID NO: 22 - protein, 240 aa, artificial
  IPT isopentenyl transferase IPT (E88G)
MDLRLIFGPTCTGKTSTAVALAQQTGLPVLSLDRVQCCPQLSTGSGRPTVEELKGTSRLYLDDRPLVKGIIAAKQAHERLMGEVYNYEAHGGLILEGGSISLLKCMAQSSYWSADFRWHIIRHELAHEETFMNVAKARVKQMLRPASGLSIIQELVDLWKEPRLRRILKEIDGYRYAMLFVSQNQITSDMLLQLDADMEDKLIHGIAQEYLIHARRQEQKFPRVNAAAYDGFEGHPFGMY

SEQ ID NO: 23 - DNA, 513 bp, Tomato
atggcaaatccaaaggttttctttgaccttaccatcggtggtgcaccagctggtcgtgtggtgatggagctcttcgccgataccactcccaaaaccgctgagaacttccgagctctttgtaccggtgagaaaggtgttggaaagatggggaagcctttgcactacaagggctcaaccttccaccgtgtgatcccagggttcatgtgtcaaggaggtgatttcaccgccggaaacgggaccggaggagagtcgatctatggagccaaattcaacgatgagaacttcgttaagaagcacaccggccctggaatcctctccatggctaatgctggacctggaaccaacggttctcagtttttcatctgtaccgctaagactgagtggctcaacggaaagcacgtcgtgtttggacaagttgttgaaggcatggatgtgattaagaaggcagaggctgttggatctagctctggaaggtgctccaagcctgtggttattgctgactgcggtcaactc

SEQ ID NO: 24 - protein, 171 aa, Tomato
MANPKVFFDLTIGGAPAGRVVMELFADTTPKTAENFRALCTGEKGVGKMGKPLHYKGSTFHRVIPGFMCQGGDFTAGNGTGGESIYGAKFNDENFVKKHTGPGILSMANAGPGTNGSQFFICTAKTEWLNGKHVVFGQVVEGMDVIKKAEAVGSSSGRCSKPVVIADCGQL

SEQ ID NO: 25 - DNA, 4780 bp, artificial
  nucleotide sequence of T-DNA o fpNMD2492
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataccatgggagaccattaagttggtaatttgaggtctcttaaggatcctctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcaggcatgccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctctatttttacaacaattaccaacaacaacaaacaacaaacaacattacaattacatttacaattaccatggaacgagctatacaaggaaacgatgctagggaacaagcttatggtgaacgttggaatggaggatcaggaagttccacttctcccttcaaacttcctgacgaaagtccgagttggactgagtggcggctacataacgatgagacgatttcgaatcaagataatccccttggtttcaaggaaagctggggtttcgggaaagttgtatttaagagatatctcagatacgacgggacggaaacttcactgcacagagtccttggatcttggacgggagattcggttaactatgcagcatctcgatttctcggtttcgaccagatcggatgtacctatagtattcggtttcgaggagttagtgtcaccatttctggagggtcgcgaactcttcagcatctcagtgaaatggcaattcggtctaagcaagaactgctacagcttaccccagtcaaagtggaaagtgatgtatcaagaggatgccctgaaggtgttgaaaccttcgaagaagaaagcgagtaaggatcctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcataagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 26 - DNA, 9656 bp, artificial
  T-DNA of pNMD035
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctagcttggaattggtaccacgcgtttcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaagttttagttttattgcaacaacaacaacaaattacaataacaacaaacaaaatacaaacaacaacaacatggcacaatttcaacaaacaattgacatgcaaactctccaagccgctgcgggacgcaacagcttggtgaatgatttggcatctcgtcgcgtttacgataatgcagtcgaggagctgaatgctcgttccagacgtcccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcaggtccacttctccaaggcagtgtctacggaacagaccctgattgcaacaaacgcatatccggagttcgagatttcctttactcatacgcaatccgctgtgcactccttggccggaggccttcggtcacttgagttggagtatctcatgatgcaagttccgttcggttctctgacgtacgacatcggcggtaacttttccgcgcaccttttcaaagggcgcgattacgttcactgctgcatgcctaatctggatgtacgtgacattgctcgccatgaaggacacaaggaagctatttacagttatgtgaatcgtttgaaaaggcagcagcgtcctgtgcctgaataccagagggcagctttcaacaactacgctgagaacccgcacttcgtccattgcgacaaacctttccaacagtgtgaattgacgacagcgtatggcactgacacctacgctgtagctctccatagcatttatgatatccctgttgaggagttcggttctgcgctactcaggaagaatgtgaaaacttgtttcgcggcctttcatttccatgagaatatgcttctagattgtgatacagtcacactcgatgagattggagctacgttccagaaatcaggtaacattccttagttacctttcttttctttttccatcataagtttatagattgtacatgctttgagatttttctttgcaaacaatctcaggtgataacctgagcttcttcttccataatgagagcactctcaattacacccacagcttcagcaacatcatcaagtacgtgtgcaagacgttcttccctgctagtcaacgcttcgtgtaccacaaggagttcctggtcactagagtcaacacttggtactgcaagttcacgagagtggatacgttcactctgttccgtggtgtgtaccacaacaatgtggattgcgaagagttttacaaggctatggacgatgcgtggcactacaaaaagacgttagcaatgcttaatgccgagaggaccatcttcaaggataacgctgcgttaaacttctggttcccgaaggtgctcttgaaattggaagtcttcttttgttgtctaaacctatcaatttctttgcggaaatttatttgaagctgtagagttaaaattgagtcttttaaacttttgtaggtgagagacatggttatcgtccctctctttgacgcttctatcacaactggtaggatgtctaggagagaggttatggtgaacaaggacttcgtctacacggtcctaaatcacatcaagacctatcaagctaaggcactgacgtacgcaaacgtgctgagcttcgtggagtctattaggtctagagtgataattaacggtgtcactgccaggtaagttgttacttatgattgttttcctctctgctacatgtattttgttgttcatttctgtaagatataagaattgagttttcctctgatgatattattaggtctgaatgggacacagacaaggcaattctaggtccattagcaatgacattcttcctgatcacgaagctgggtcatgtgcaagatgaaataatcctgaaaaagttccagaagttcgacagaaccaccaatgagctgatttggacaagtctctgcgatgccctgatgggggttattccctcggtcaaggagacgcttgtgcgcggtggttttgtgaaagtagcagaacaagccttagagatcaaggttagtatcatatgaagaaatacctagtttcagttgatgaatgctattttctgacctcagttgttctcttttgagaattatttcttttctaatttgcctgatttttctattaattcattaggttcccgagctatactgtaccttcgccgaccgattggtactacagtacaagaaggcggaggagttccaatcgtgtgatctttccaaacctctagaagagtcagagaagtactacaacgcattatccgagctatcagtgcttgagaatctcgactcttttgacttagaggcgtttaagactttatgtcagcagaagaatgtggacccggatatggcagcaaaggtaaatcctggtccacacttttacgataaaaacacaagattttaaactatgaactgatcaataatcattcctaaaagaccacacttttgttttgtttctaaagtaatttttactgttataacaggtggtcgtagcaatcatgaagtcagaattgacgttgcctttcaagaaacctacagaagaggaaatctcggagtcgctaaaaccaggagaggggtcgtgtgcagagcataaggaagtgttgagcttacaaaatgatgctccgttcccgtgtgtgaaaaatctagttgaaggttccgtgccggcgtatggaatgtgtcctaagggtggtggtttcgacaaattggatgtggacattgctgatttccatctcaagagtgtagatgcagttaaaaagggaactatgatgtctgcggtgtacacagggtctatcaaagttcaacaaatgaagaactacatagattacttaagtgcgtcgctggcagctacagtctcaaacctctgcaaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcttagagatgttcacggcgttgacccagagtcacaggagaaatctggagtgtgggatgttaggagaggacgttggttacttaaacctaatgcgaaaagtcacgcgtggggtgtggcagaagacgccaaccacaagttggttattgtgttactcaactgggatgacggaaagccggtttgtgatgagacatggttcagggtggcggtgtcaagcgattccttgatatattcggatatgggaaaacttaagacgctcacgtcttgcagtccaaatggtgagccaccggagcctaacgccaaagtaattttggtcgatggtgttcccggttgtggaaaaacgaaggagattatcgaaaaggtaagttctgcatttggttatgctccttgcattttaggtgttcgtcgctcttccatttccatgaatagctaagattttttttctctgcattcattcttcttgcctcagttctaactgtttgtggtatttttgttttaattattgctacaggtaaacttctctgaagacttgattttagtccctgggaaggaagcttctaagatgatcatccggagggccaaccaagctggtgtgataagagcggataaggacaatgttagaacggtggattccttcttgatgcatccttctagaagggtgtttaagaggttgtttatcgatgaaggactaatgctgcatacaggttgtgtaaatttcctactgctgctatctcaatgtgacgtcgcatatgtgtatggggacacaaagcaaattccgttcatttgcagagtcgcgaactttccgtatccagcgcattttgcaaaactcgtcgctgatgagaaggaagtcagaagagttacgctcaggtaaagcaactgtgttttaatcaatttcttgtcaggatatatggattataacttaatttttgagaaatctgtagtatttggcgtgaaatgagtttgctttttggtttctcccgtgttataggtgcccggctgatgttacgtatttccttaacaagaagtatgacggggcggtgatgtgtaccagcgcggtagagagatccgtgaaggcagaagtggtgagaggaaagggtgcattgaacccaataaccttaccgttggagggtaaaattttgaccttcacacaagctgacaagttcgagttactggagaagggttacaaggtaaagtttccaactttcctttaccatatcaaactaaagttcgaaactttttatttgatcaacttcaaggccacccgatctttctattcctgattaatttgtgatgaatccatattgacttttgatggttacgcaggatgtgaacactgtgcacgaggtgcaaggggagacgtacgagaagactgctattgtgcgcttgacatcaactccgttagagatcatatcgagtgcgtcacctcatgttttggtggcgctgacaagacacacaacgtgttgtaaatattacaccgttgtgttggacccgatggtgaatgtgatttcagaaatggagaagttgtccaatttccttcttgacatgtatagagttgaagcaggtctgtctttcctatttcatatgtttaatcctaggaatttgatcaattgattgtatgtatgtcgatcccaagactttcttgttcacttatatcttaactctctctttgctgtttcttgcaggtgtccaatagcaattacaaatcgatgcagtattcaggggacagaacttgtttgttcagacgcccaagtcaggagattggcgagatatgcaattttactatgacgctcttcttcccggaaacagtactattctcaatgaatttgatgctgttacgatgaatttgagggatatttccttaaacgtcaaagattgcagaatcgacttctccaaatccgtgcaacttcctaaagaacaacctattttcctcaagcctaaaataagaactgcggcagaaatgccgagaactgcaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtttgctggaaaatttggttgcaatgatcaaaagaaacatgaatgcgccggatttgacagggacaattgacattgaggatactgcatctctggtggttgaaaagttttgggattcgtatgttgacaaggaatttagtggaacgaacgaaatgaccatgacaagggagagcttctccaggtaaggacttctcatgaatattagtggcagattagtgttgttaaagtctttggttagataatcgatgcctcctaattgtccatgttttactggttttctacaattaaaggtggctttcgaaacaagagtcatctacagttggtcagttagcggactttaactttgtggatttgccggcagtagatgagtacaagcatatgatcaagagtcaaccaaagcaaaagttagacttgagtattcaagacgaatatcctgcattgcagacgatagtctaccattcgaaaaagatcaatgcgattttcggtccaatgttttcagaacttacgaggatgttactcgaaaggattgactcttcgaagtttctgttctacaccagaaagacacctgcacaaatagaggacttcttttctgacctagactcaacccaggcgatggaaattctggaactcgacatttcgaagtacgataagtcacaaaacgagttccattgtgctgtagagtacaagatctgggaaaagttaggaattgatgagtggctagctgaggtctggaaacaaggtgagttcctaagttccatttttttgtaatccttcaatgttattttaacttttcagatcaacatcaaaattaggttcaattttcatcaaccaaataatatttttcatgtatatataggtcacagaaaaacgaccttgaaagattatacggccggaatcaaaacatgtctttggtatcaaaggaaaagtggtgatgtgacaacctttattggtaataccatcatcattgccgcatgtttgagctcaatgatccccatggacaaagtgataaaggcagctttttgtggagacgatagcctgatttacattcctaaaggtttagacttgcctgatattcaggcgggcgcgaacctcatgtggaacttcgaggccaaactcttcaggaagaagtatggttacttctgtggtcgttatgttattcaccatgatagaggagccattgtgtattacgatccgcttaaactaatatctaagttaggttgtaaacatattagagatgttgttcacttagaagagttacgcgagtctttgtgtgatgtagctagtaacttaaataattgtgcgtatttttcacagttagatgaggccgttgccgaggttcataagaccgcggtaggcggttcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctaggagccgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggtaaggatttttatgatatagtatgcttatgtattttgtactgaaagcatatcctgcttcattgggatattactgaaagcatttaactacatgtaaactcacttgatgatcaataaacttgattttgcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtaagccatcttcctgcttatttttataatgaacatagaaataggaagttgtgcagagaaactaattaacctgactcaaaatctaccctcataattgttgtttgatattggtcttgtattttgcaggtgtggttgacgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaacgtcttacaacattacaaacccgaatcagtaccagtatttcgataagaaacaagaaaccatgagagacctgatatccacaaccgtggtctcgagcttactagagcgtggtgcgcacgatagcgcatagtgtttttctctccacttgaatcgaagagatagacttacggtgtaaatccgtaggggtggcgtaaaccaaattacgcaatgttttgggttccatttaaatcgaaaccccttatttcctggatcacctgttaacgcacgtttgacgtgtattacagtgggaataagtaaaagtgagaggttcgaatcctccctaaccccgggtaggggcccagcggccgctctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 27 - DNA, 9711 bp, artificial
  T-DNA of pNMD661
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctagcttggaattggtaccacgcgtttcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaagttttagttttattgcaacaacaacaacaaattacaataacaacaaacaaaatacaaacaacaacaacatggcacaatttcaacaaacaattgacatgcaaactctccaagccgctgcgggacgcaacagcttggtgaatgatttggcatctcgtcgcgtttacgataatgcagtcgaggagctgaatgctcgttccagacgtcccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcaggtccacttctccaaggcagtgtctacggaacagaccctgattgcaacaaacgcatatccggagttcgagatttcctttactcatacgcaatccgctgtgcactccttggccggaggccttcggtcacttgagttggagtatctcatgatgcaagttccgttcggttctctgacgtacgacatcggcggtaacttttccgcgcaccttttcaaagggcgcgattacgttcactgctgcatgcctaatctggatgtacgtgacattgctcgccatgaaggacacaaggaagctatttacagttatgtgaatcgtttgaaaaggcagcagcgtcctgtgcctgaataccagagggcagctttcaacaactacgctgagaacccgcacttcgtccattgcgacaaacctttccaacagtgtgaattgacgacagcgtatggcactgacacctacgctgtagctctccatagcatttatgatatccctgttgaggagttcggttctgcgctactcaggaagaatgtgaaaacttgtttcgcggcctttcatttccatgagaatatgcttctagattgtgatacagtcacactcgatgagattggagctacgttccagaaatcaggtaacattccttagttacctttcttttctttttccatcataagtttatagattgtacatgctttgagatttttctttgcaaacaatctcaggtgataacctgagcttcttcttccataatgagagcactctcaattacacccacagcttcagcaacatcatcaagtacgtgtgcaagacgttcttccctgctagtcaacgcttcgtgtaccacaaggagttcctggtcactagagtcaacacttggtactgcaagttcacgagagtggatacgttcactctgttccgtggtgtgtaccacaacaatgtggattgcgaagagttttacaaggctatggacgatgcgtggcactacaaaaagacgttagcaatgcttaatgccgagaggaccatcttcaaggataacgctgcgttaaacttctggttcccgaaggtgctcttgaaattggaagtcttcttttgttgtctaaacctatcaatttctttgcggaaatttatttgaagctgtagagttaaaattgagtcttttaaacttttgtaggtgagagacatggttatcgtccctctctttgacgcttctatcacaactggtaggatgtctaggagagaggttatggtgaacaaggacttcgtctacacggtcctaaatcacatcaagacctatcaagctaaggcactgacgtacgcaaacgtgctgagcttcgtggagtctattaggtctagagtgataattaacggtgtcactgccaggtaagttgttacttatgattgttttcctctctgctacatgtattttgttgttcatttctgtaagatataagaattgagttttcctctgatgatattattaggtctgaatgggacacagacaaggcaattctaggtccattagcaatgacattcttcctgatcacgaagctgggtcatgtgcaagatgaaataatcctgaaaaagttccagaagttcgacagaaccaccaatgagctgatttggacaagtctctgcgatgccctgatgggggttattccctcggtcaaggagacgcttgtgcgcggtggttttgtgaaagtagcagaacaagccttagagatcaaggttagtatcatatgaagaaatacctagtttcagttgatgaatgctattttctgacctcagttgttctcttttgagaattatttcttttctaatttgcctgatttttctattaattcattaggttcccgagctatactgtaccttcgccgaccgattggtactacagtacaagaaggcggaggagttccaatcgtgtgatctttccaaacctctagaagagtcagagaagtactacaacgcattatccgagctatcagtgcttgagaatctcgactcttttgacttagaggcgtttaagactttatgtcagcagaagaatgtggacccggatatggcagcaaaggtaaatcctggtccacacttttacgataaaaacacaagattttaaactatgaactgatcaataatcattcctaaaagaccacacttttgttttgtttctaaagtaatttttactgttataacaggtggtcgtagcaatcatgaagtcagaattgacgttgcctttcaagaaacctacagaagaggaaatctcggagtcgctaaaaccaggagaggggtcgtgtgcagagcataaggaagtgttgagcttacaaaatgatgctccgttcccgtgtgtgaaaaatctagttgaaggttccgtgccggcgtatggaatgtgtcctaagggtggtggtttcgacaaattggatgtggacattgctgatttccatctcaagagtgtagatgcagttaaaaagggaactatgatgtctgcggtgtacacagggtctatcaaagttcaacaaatgaagaactacatagattacttaagtgcgtcgctggcagctacagtctcaaacctctgcaaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcttagagatgttcacggcgttgacccagagtcacaggagaaatctggagtgtgggatgttaggagaggacgttggttacttaaacctaatgcgaaaagtcacgcgtggggtgtggcagaagacgccaaccacaagttggttattgtgttactcaactgggatgacggaaagccggtttgtgatgagacatggttcagggtggcggtgtcaagcgattccttgatatattcggatatgggaaaacttaagacgctcacgtcttgcagtccaaatggtgagccaccggagcctaacgccaaagtaattttggtcgatggtgttcccggttgtggaaaaacgaaggagattatcgaaaaggtaagttctgcatttggttatgctccttgcattttaggtgttcgtcgctcttccatttccatgaatagctaagattttttttctctgcattcattcttcttgcctcagttctaactgtttgtggtatttttgttttaattattgctacaggtaaacttctctgaagacttgattttagtccctgggaaggaagcttctaagatgatcatccggagggccaaccaagctggtgtgataagagcggataaggacaatgttagaacggtggattccttcttgatgcatccttctagaagggtgtttaagaggttgtttatcgatgaaggactaatgctgcatacaggttgtgtaaatttcctactgctgctatctcaatgtgacgtcgcatatgtgtatggggacacaaagcaaattccgttcatttgcagagtcgcgaactttccgtatccagcgcattttgcaaaactcgtcgctgatgagaaggaagtcagaagagttacgctcaggtaaagcaactgtgttttaatcaatttcttgtcaggatatatggattataacttaatttttgagaaatctgtagtatttggcgtgaaatgagtttgctttttggtttctcccgtgttataggtgcccggctgatgttacgtatttccttaacaagaagtatgacggggcggtgatgtgtaccagcgcggtagagagatccgtgaaggcagaagtggtgagaggaaagggtgcattgaacccaataaccttaccgttggagggtaaaattttgaccttcacacaagctgacaagttcgagttactggagaagggttacaaggtaaagtttccaactttcctttaccatatcaaactaaagttcgaaactttttatttgatcaacttcaaggccacccgatctttctattcctgattaatttgtgatgaatccatattgacttttgatggttacgcaggatgtgaacactgtgcacgaggtgcaaggggagacgtacgagaagactgctattgtgcgcttgacatcaactccgttagagatcatatcgagtgcgtcacctcatgttttggtggcgctgacaagacacacaacgtgttgtaaatattacaccgttgtgttggacccgatggtgaatgtgatttcagaaatggagaagttgtccaatttccttcttgacatgtatagagttgaagcaggtctgtctttcctatttcatatgtttaatcctaggaatttgatcaattgattgtatgtatgtcgatcccaagactttcttgttcacttatatcttaactctctctttgctgtttcttgcaggtgtccaatagcaattacaaatcgatgcagtattcaggggacagaacttgtttgttcagacgcccaagtcaggagattggcgagatatgcaattttactatgacgctcttcttcccggaaacagtactattctcaatgaatttgatgctgttacgatgaatttgagggatatttccttaaacgtcaaagattgcagaatcgacttctccaaatccgtgcaacttcctaaagaacaacctattttcctcaagcctaaaataagaactgcggcagaaatgccgagaactgcaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtttgctggaaaatttggttgcaatgatcaaaagaaacatgaatgcgccggatttgacagggacaattgacattgaggatactgcatctctggtggttgaaaagttttgggattcgtatgttgacaaggaatttagtggaacgaacgaaatgaccatgacaagggagagcttctccaggtaaggacttctcatgaatattagtggcagattagtgttgttaaagtctttggttagataatcgatgcctcctaattgtccatgttttactggttttctacaattaaaggtggctttcgaaacaagagtcatctacagttggtcagttagcggactttaactttgtggatttgccggcagtagatgagtacaagcatatgatcaagagtcaaccaaagcaaaagttagacttgagtattcaagacgaatatcctgcattgcagacgatagtctaccattcgaaaaagatcaatgcgattttcggtccaatgttttcagaacttacgaggatgttactcgaaaggattgactcttcgaagtttctgttctacaccagaaagacacctgcacaaatagaggacttcttttctgacctagactcaacccaggcgatggaaattctggaactcgacatttcgaagtacgataagtcacaaaacgagttccattgtgctgtagagtacaagatctgggaaaagttaggaattgatgagtggctagctgaggtctggaaacaaggtgagttcctaagttccatttttttgtaatccttcaatgttattttaacttttcagatcaacatcaaaattaggttcaattttcatcaaccaaataatatttttcatgtatatataggtcacagaaaaacgaccttgaaagattatacggccggaatcaaaacatgtctttggtatcaaaggaaaagtggtgatgtgacaacctttattggtaataccatcatcattgccgcatgtttgagctcaatgatccccatggacaaagtgataaaggcagctttttgtggagacgatagcctgatttacattcctaaaggtttagacttgcctgatattcaggcgggcgcgaacctcatgtggaacttcgaggccaaactcttcaggaagaagtatggttacttctgtggtcgttatgttattcaccatgatagaggagccattgtgtattacgatccgcttaaactaatatctaagttaggttgtaaacatattagagatgttgttcacttagaagagttacgcgagtctttgtgtgatgtagctagtaacttaaataattgtgcgtatttttcacagttagatgaggccgttgccgaggttcataagaccgcggtaggcggttcgtttgctttttgtagtataattaagtatttgtcagataagagattgtttagagatttgttctttgtttgataatgtcgatagtctcgtacgaacctaaggtgagtgatttcctcaatctttcgaagaaggaagagatcttgccgaaggctctaacgaggttaaaaaccgtgtctattagtactaaagatattatatctgtcaaggagtcggagactttgtgtgatatagatttgttaatcaatgtgccattagataagtatagatatgtgggtatcctagctaggagccgtttttaccggagagtggctagtgccagacttcgttaaaggtggagtgacgataagtgtgatagataagcgtctggtgaactcaaaggagtgcgtgattggtacgtacagagccgcagccaagagtaagaggttccagttcaaattggttccaaattactttgtgtccaccgtggacgcaaagaggaagccgtggcaggtaaggatttttatgatatagtatgcttatgtattttgtactgaaagcatatcctgcttcattgggatattactgaaagcatttaactacatgtaaactcacttgatgatcaataaacttgattttgcaggttcatgttcgtatacaagacttgaagattgaggcgggttggcagccgttagctctggaagtagtttcagttgctatggtcaccaataacgttgtcatgaagggtttgagggaaaaggtcgtcgcaataaatgatccggacgtcgaaggtttcgaaggtaagccatcttcctgcttatttttataatgaacatagaaataggaagttgtgcagagaaactaattaacctgactcaaaatctaccctcataattgttgtttgatattggtcttgtattttgcaggtgtggttgacgaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaacgtcttacaacattacaaacccgaataatcgataactcgagtatttttacaacaattaccaacaacaacaaacaacaaacaacattacaattacatttacaattatccatgtgagaccccacaaccgtggggtctcagatcagcggcccctagagcgtggtgcgcacgatagcgcatagtgtttttctctccacttgaatcgaagagatagacttacggtgtaaatccgtaggggtggcgtaaaccaaattacgcaatgttttgggttccatttaaatcgaaaccccttatttcctggatcacctgttaacgcacgtttgacgtgtattacagtgggaataagtaaaagtgagaggttcgaatcctccctaaccccgggtaggggcccagcggccgctctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 28 - DNA, 7422 bp, artificial
  T-DNA of pNMD670
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatcctaccacaagaacctgttacatttatgtttcttaaacccagaaagctaaactacatgagaagaaacccgcggatcaaggacattttccaaaatgttgccattgaaccaagagacgtagccaggtaccccaaggaaacaataattgacaaactcacagagatcacaacggaaacagcatacattagtgacactctgcacttcttggatccgagctacatagtggagacattccaaaactgcccaaaattgcaaacattgtatgcgaccttagttctccccgttgaggcagcctttaaaatggaaagcactcacccgaacatatacagcctcaaatacttcggagatggtttccagtatataccaggcaaccatggtggcggggcataccatcatgaattcgctcatctacaatggctcaaagtgggaaagatcaagtggagggaccccaaggatagctttctcggacatctcaattacacgactgagcaggttgagatgcacacagtgacagtacagttgcaggaatcgttcgcggcaaaccacttgtactgcatcaggagaggagacttgctcacaccggaggtgcgcactttcggccaacctgacaggtacgtgattccaccacagatcttcctcccaaaagttcacaactgcaagaagccgattctcaagaaaactatgatgcagctcttcttgtatgttaggacagtcaaggtcgcaaaaaattgtgacatttttgccaaagtcagacaattaattaaatcatctgacttggacaaatactctgctgtggaactggtttacttagtaagctacatggagttccttgccgatttacaagctaccacctgcttctcagacacactttctggtggcttgctaacaaagacccttgcaccggtgagggcttggatacaagagaaaaagatgcagctgtttggtcttgaggactacgcgaagttagtcaaagcagttgatttccacccggtggatttttctttcaaagtggaaacttgggacttcagattccaccccttgcaagcgtggaaagccttccgaccaagggaagtgtcggatgtagaggaaatggaaagtttgttctcagatggggacctgcttgattgcttcacaagaatgccagcttatgcggtaaacgcagaggaagatttagctgcaatcaggaaaacgcccgagatggatgtcggtcaagaagttaaagagcctgcaggagacagaaatcaatactcaaaccctgcagaaactttcctcaacaagctccacaggaaacacagtagggaggtgaaacaccaggccgcaaagaaagctaaacgcctagctgaaatccaggagtcaatgagagctgaaggtgatgccgaaccaaatgaaataagcgggacgatgggggcaatacccagcaacgccgaacttcctggcacgaatgatgccagacaagaactcacactcccaaccactaaacctgtccctgcaaggtgggaagatgcttcattcacagattctagtgtggaagaggagcaggttaaactccttggaaaagaaaccgttgaaacagcgacgcaacaagtcatcgaaggacttccttggaaacactggattcctcaattaaatgctgttggattcaaggcgctggaaattcagagggataggagtggaacaatgatcatgcccatcacagaaatggtgtccgggctggaaaaagaggacttccctgaaggaactccaaaagagttggcacgagaattgttcgctatgaacagaagccctgccaccatccctttggacctgcttagagccagagactacggcagtgatgtaaagaacaagagaattggtgccatcacaaagacacaggcaacgagttggggcgaatacttgacaggaaagatagaaagcttaactgagaggaaagttgcgacttgtgtcattcatggagctggaggttctggaaaaagtcatgccatccagaaggcattgagagaaattggcaagggctcggacatcactgtagtcctgccgaccaatgaactgcggctagattggagtaagaaagtgcctaacactgagccctatatgttcaagacctctgaaaaggcgttaattgggggaacaggcagcatagtcatctttgacgattactcaaaacttcctcccggttacatagaagccttagtctgtttctactctaaaatcaagctaatcattctaacaggagatagcagacaaagcgtctaccatgaaactgctgaggacgcctccatcaggcatttgggaccagcaacagagtacttctcaaaatactgccgatactatctcaatgccacacaccgcaacaagaaagatcttgcgaacatgcttggtgtctacagtgagagaacgggagtcaccgaaatcagcatgagcgccgagttcttagaaggaatcccaactttggtaccctcggatgagaagagaaagctgtacatgggcaccgggaggaatgacacgttcacatacgctggatgccaggggctaactaagccgaaggtacaaatagtgttggaccacaacacccaagtgtgtagcgcgaatgtgatgtacacggcactttctagagccaccgataggattcacttcgtgaacacaagtgcaaattcctctgccttctgggaaaagttggacagcaccccttacctcaagactttcctatcagtggtgagagaacaagcactcagggagtacgagccggcagaggcagagccaattcaagagcctgagccccagacacacatgtgtgtcgagaatgaggagtccgtgctagaagagtacaaagaggaactcttggaaaagtttgacagagagatccactctgaatcccatggtcattcaaactgtgtccaaactgaagacacaaccattcagttgttttcgcatcaacaagcaaaagatgagactctcctctgggcgactatagatgcgcggctcaagaccagcaatcaagaaacaaacttccgagaattcctgagcaagaaggacattggggacgttctgtttttaaactaccaaaaagctatgggtttacccaaagagcgtattcctttttcccaagaggtctgggaagcttgtgcccacgaagtacaaagcaagtacctcagcaagtcaaagtgcaacttgatcaatgggactgtgagacagagcccagacttcgatgaaaataagattatggtattcctcaagtcgcagtgggtcacaaaggtggaaaaactaggtctacccaagattaagccaggtcaaaccatagcagccttttaccagcagactgtgatgctttttggaactatggctaggtacatgcgatggttcagacaggctttccagccaaaagaagtcttcataaactgtgagacgacgccagatgacatgtctgcatgggccttgaacaactggaatttcagcagacctagcttggctaatgactacacagctttcgaccagtctcaggatggagccatgttgcaatttgaggtgctcaaagccaaacaccactgcataccagaggaaatcattcaggcatacatagatattaagactaatgcacagattttcctaggcacgttatcaattatgcgcctgactggtgaaggtcccacttttgatgcaaacactgagtgcaacatagcttacacccatacaaagtttgacatcccagccggaactgctcaagtttatgcaggagacgactccgcactggactgtgttccagaagtgaagcatagtttccacaggcttgaggacaaattactcctaaagtcaaagcctgtaatcacgcagcaaaagaagggcagttggcctgagttttgtggttggctgatcacaccaaaaggggtgatgaaagacccaattaagctccatgttagcttaaaattggctgaagctaagggtgaactcaagaaatgtcaagattcctatgaaattgatctgagttatgcctatgaccacaaggactctctgcatgacttgttcgatgagaaacagtgtcaggcacacacactcacttgcagaacactaatcaagtcagggagaggcactgtctcactttcccgcctcagaaactttctttaaccgttaagttaccttagagatttgaataagatgtcagcaccagctagtacaacacagcccatagggtcaactacctcaactaccacaaaaactgcaggcgcaactcctgccacagcttcaggcctgttcactatcccggatggggatttctttagtacagcccgtgccatagtagccagcaatgctgtcgcaacaaatgaggacctcagcaagattgaggctatttggaaggacatgaaggtgcccacagacactatggcacaggctgcttgggacttagtcagacactgtgctgatgtaggatcatccgctcaaacagaaatgatagatacaggtccctattccaacggcatcagcagagctagactggcagcagcaattaaagaggtgtgcacacttaggcaattttgcatgaagtatgccccagtggtatggaactggatgttaactaacaacagtccacctgctaactggcaagcacaaggtttcaagcctgagcacaaattcgctgcattcgacttcttcaatggagtcaccaacccagctgccatcatgcccaaagaggggctcatccggccaccgtctgaagctgaaatgaatgctgcccaaactgctgcctttgtgaagattacaaaggccagggcacaatccaacgactttgccagcctagatgcagctgtcactcgaggaaggatcaccggaacgaccacagcagaggcagtcgttactctgcctcctccataacagaaactttctttaaccgttaagttaccttagagatttgaataagatggatattctcatcagtagtttgaaaagtttaggttattctaggacttccaaatctttagattcaggacctttggtagtacatgcagtagccggagccggtaagtccacagccctaaggaagttgatcctcagacacccaacattcaccgtgcatacactcggtgtccctgacaaggtgagtatcagaactagaggcatacagaagccaggacctattcctgagggcaacttcgcaatcctcgatgagtatactttggacaacaccacaaggaactcataccaggcactttttgctgacccttatcaggcaccggagtttagcctagagccccacttctacttggaaacatcatttcgagttccgaggaaagtggcagatttgatagctggctgtggcttcgatttcgagacgaactcaccggaagaagggcacttagagatcactggcatattcaaagggcccctactcggaaaggtgatagccattgatgaggagtctgagacaacactgtccaggcatggtgttgagtttgttaagccctgccaagtgacgggacttgagttcaaagtagtcactattgtgtctgccgcaccaatagaggaaattggccagtccacagctttctacaacgctatcaccaggtcaaagggattgacatatgtccgcgcagggccataggctgaccgctccggtcaattctgaaaaagtgtacatagtattaggtctatcatttgctttagtttcaattacctttctgctttctagaaatagcttaccccacgtcggtgacaacattcacagcttgccacacggaggagcttacagagacggcaccaaagcaatcttgtacaactccccaaatctagggtcacgagtgagtctacacaacggaaagaacgcagcatttgctgccgttttgctactgactttgctgatctatggaagtaaatacatatctcaacgcaatcatacttgtgcttgtggtaacaatcatagcagtcattagcacttccttagtgaggactgaaccttgtgtcatcaagattactggggaatcaatcacagtgttggcttgcaaactagatgcagaaaccataagggccattgccgatctcaagccactctccgttgaacggttaagtttccattgatactcgaaagaggtcagcaccagctagcaacaaacaagaacatgagagacctcgcgatttaaatcgatggtctcagatcggtcgtatcactggaacaacaaccgctgaggctgttgtcactctaccaccaccataactacgtctacataaccgacgcctaccccagtttcatagtattttctggtttgattgtatgaataatataaataaaaaaaaaaaaaaaaaaaaaaaactagtgagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 29 - DNA, 6662 bp, artificial
  T-DNA of pNMD694
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatcctaccacaagaacctgttacatttatgtttcttaaacccagaaagctaaactacatgagaagaaacccgcggatcaaggacattttccaaaatgttgccattgaaccaagagacgtagccaggtaccccaaggaaacaataattgacaaactcacagagatcacaacggaaacagcatacattagtgacactctgcacttcttggatccgagctacatagtggagacattccaaaactgcccaaaattgcaaacattgtatgcgaccttagttctccccgttgaggcagcctttaaaatggaaagcactcacccgaacatatacagcctcaaatacttcggagatggtttccagtatataccaggcaaccatggtggcggggcataccatcatgaattcgctcatctacaatggctcaaagtgggaaagatcaagtggagggaccccaaggatagctttctcggacatctcaattacacgactgagcaggttgagatgcacacagtgacagtacagttgcaggaatcgttcgcggcaaaccacttgtactgcatcaggagaggagacttgctcacaccggaggtgcgcactttcggccaacctgacaggtacgtgattccaccacagatcttcctcccaaaagttcacaactgcaagaagccgattctcaagaaaactatgatgcagctcttcttgtatgttaggacagtcaaggtcgcaaaaaattgtgacatttttgccaaagtcagacaattaattaaatcatctgacttggacaaatactctgctgtggaactggtttacttagtaagctacatggagttccttgccgatttacaagctaccacctgcttctcagacacactttctggtggcttgctaacaaagacccttgcaccggtgagggcttggatacaagagaaaaagatgcagctgtttggtcttgaggactacgcgaagttagtcaaagcagttgatttccacccggtggatttttctttcaaagtggaaacttgggacttcagattccaccccttgcaagcgtggaaagccttccgaccaagggaagtgtcggatgtagaggaaatggaaagtttgttctcagatggggacctgcttgattgcttcacaagaatgccagcttatgcggtaaacgcagaggaagatttagctgcaatcaggaaaacgcccgagatggatgtcggtcaagaagttaaagagcctgcaggagacagaaatcaatactcaaaccctgcagaaactttcctcaacaagctccacaggaaacacagtagggaggtgaaacaccaggccgcaaagaaagctaaacgcctagctgaaatccaggagtcaatgagagctgaaggtgatgccgaaccaaatgaaataagcgggacgatgggggcaatacccagcaacgccgaacttcctggcacgaatgatgccagacaagaactcacactcccaaccactaaacctgtccctgcaaggtgggaagatgcttcattcacagattctagtgtggaagaggagcaggttaaactccttggaaaagaaaccgttgaaacagcgacgcaacaagtcatcgaaggacttccttggaaacactggattcctcaattaaatgctgttggattcaaggcgctggaaattcagagggataggagtggaacaatgatcatgcccatcacagaaatggtgtccgggctggaaaaagaggacttccctgaaggaactccaaaagagttggcacgagaattgttcgctatgaacagaagccctgccaccatccctttggacctgcttagagccagagactacggcagtgatgtaaagaacaagagaattggtgccatcacaaagacacaggcaacgagttggggcgaatacttgacaggaaagatagaaagcttaactgagaggaaagttgcgacttgtgtcattcatggagctggaggttctggaaaaagtcatgccatccagaaggcattgagagaaattggcaagggctcggacatcactgtagtcctgccgaccaatgaactgcggctagattggagtaagaaagtgcctaacactgagccctatatgttcaagacctctgaaaaggcgttaattgggggaacaggcagcatagtcatctttgacgattactcaaaacttcctcccggttacatagaagccttagtctgtttctactctaaaatcaagctaatcattctaacaggagatagcagacaaagcgtctaccatgaaactgctgaggacgcctccatcaggcatttgggaccagcaacagagtacttctcaaaatactgccgatactatctcaatgccacacaccgcaacaagaaagatcttgcgaacatgcttggtgtctacagtgagagaacgggagtcaccgaaatcagcatgagcgccgagttcttagaaggaatcccaactttggtaccctcggatgagaagagaaagctgtacatgggcaccgggaggaatgacacgttcacatacgctggatgccaggggctaactaagccgaaggtacaaatagtgttggaccacaacacccaagtgtgtagcgcgaatgtgatgtacacggcactttctagagccaccgataggattcacttcgtgaacacaagtgcaaattcctctgccttctgggaaaagttggacagcaccccttacctcaagactttcctatcagtggtgagagaacaagcactcagggagtacgagccggcagaggcagagccaattcaagagcctgagccccagacacacatgtgtgtcgagaatgaggagtccgtgctagaagagtacaaagaggaactcttggaaaagtttgacagagagatccactctgaatcccatggtcattcaaactgtgtccaaactgaagacacaaccattcagttgttttcgcatcaacaagcaaaagatgagactctcctctgggcgactatagatgcgcggctcaagaccagcaatcaagaaacaaacttccgagaattcctgagcaagaaggacattggggacgttctgtttttaaactaccaaaaagctatgggtttacccaaagagcgtattcctttttcccaagaggtctgggaagcttgtgcccacgaagtacaaagcaagtacctcagcaagtcaaagtgcaacttgatcaatgggactgtgagacagagcccagacttcgatgaaaataagattatggtattcctcaagtcgcagtgggtcacaaaggtggaaaaactaggtctacccaagattaagccaggtcaaaccatagcagccttttaccagcagactgtgatgctttttggaactatggctaggtacatgcgatggttcagacaggctttccagccaaaagaagtcttcataaactgtgagacgacgccagatgacatgtctgcatgggccttgaacaactggaatttcagcagacctagcttggctaatgactacacagctttcgaccagtctcaggatggagccatgttgcaatttgaggtgctcaaagccaaacaccactgcataccagaggaaatcattcaggcatacatagatattaagactaatgcacagattttcctaggcacgttatcaattatgcgcctgactggtgaaggtcccacttttgatgcaaacactgagtgcaacatagcttacacccatacaaagtttgacatcccagccggaactgctcaagtttatgcaggagacgactccgcactggactgtgttccagaagtgaagcatagtttccacaggcttgaggacaaattactcctaaagtcaaagcctgtaatcacgcagcaaaagaagggcagttggcctgagttttgtggttggctgatcacaccaaaaggggtgatgaaagacccaattaagctccatgttagcttaaaattggctgaagctaagggtgaactcaagaaatgtcaagattcctatgaaattgatctgagttatgcctatgaccacaaggactctctgcatgacttgttcgatgagaaacagtgtcaggcacacacactcacttgcagaacactaatcaagtcagggagaggcactgtctcactttcccgcctcagaaactttctttaaccgttaagttaccttagagatttgaataagatggatattctcatcagtagtttgaaaagtttaggttattctaggacttccaaatctttagattcaggacctttggtagtacatgcagtagccggagccggtaagtccacagccctaaggaagttgatcctcagacacccaacattcaccgtgcatacactcggtgtccctgacaaggtgagtatcagaactagaggcatacagaagccaggacctattcctgagggcaacttcgcaatcctcgatgagtatactttggacaacaccacaaggaactcataccaggcactttttgctgacccttatcaggcaccggagtttagcctagagccccacttctacttggaaacatcatttcgagttccgaggaaagtggcagatttgatagctggctgtggcttcgatttcgagacgaactcaccggaagaagggcacttagagatcactggcatattcaaagggcccctactcggaaaggtgatagccattgatgaggagtctgagacaacactgtccaggcatggtgttgagtttgttaagccctgccaagtgacgggacttgagttcaaagtagtcactattgtgtctgccgcaccaatagaggaaattggccagtccacagctttctacaacgctatcaccaggtcaaagggattgacatatgtccgcgcagggccataggctgaccgctccggtcaattctgaaaaagtgtacatagtattaggtctatcatttgctttagtttcaattacctttctgctttctagaaatagcttaccccacgtcggtgacaacattcacagcttgccacacggaggagcttacagagacggcaccaaagcaatcttgtacaactccccaaatctagggtcacgagtgagtctacacaacggaaagaacgcagcatttgctgccgttttgctactgactttgctgatctatggaagtaaatacatatctcaacgcaatcatacttgtgcttgtggtaacaatcatagcagtcattagcacttccttagtgaggactgaaccttgtgtcatcaagattactggggaatcaatcacagtgttggcttgcaaactagatgcagaaaccataagggccattgccgatctcaagccactctccgttgaacggttaagtttccattgatactcgaaagaggtcagcaccagctagcaacaaacaagaacatgagagacctcgcgatttaaatcgatggtctcagatcggtcgtatcactggaacaacaaccgctgaggctgttgtcactctaccaccaccataactacgtctacataaccgacgcctaccccagtttcatagtattttctggtttgattgtatgaataatataaataaaaaaaaaaaaaaaaaaaaaaaactagtgagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 30 - DNA, 5168 bp, artificial
  T-DNA of pNMD3486
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataccatggcggatacgccttcgagcccagctggagatggcggagaaagcggcggttccgttagggagcaggatcgataccttcctatagctaatatcagcaggatcatgaagaaagcgttgcctcctaatggtaagattggaaaagatgctaaggatacagttcaggaatgcgtctctgagttcatcagcttcatcactagcgaggccagtgataagtgtcaaaaagagaaaaggaaaactgtgaatggtgatgatttgttgtgggcaatggcaacattaggatttgaggattacctggaacctctaaagatatacctagcgaggtacagggagttggagggtgataataagggatcaggaaagagtggagatggatcaaatagagatgctggtggcggtgtttctggtgaagaaatgccgagctggtaaggatcctctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcaggcatgccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctctatttttacaacaattaccaacaacaacaaacaacaaacaacattacaattacatttacaattaccatggaacgagctatacaaggaaacgatgctagggaacaagcttatggtgaacgttggaatggaggatcaggaagttccacttctcccttcaaacttcctgacgaaagtccgagttggactgagtggcggctacataacgatgagacgatttcgaatcaagataatccccttggtttcaaggaaagctggggtttcgggaaagttgtatttaagagatatctcagatacgacgggacggaaacttcactgcacagagtccttggatcttggacgggagattcggttaactatgcagcatctcgatttctcggtttcgaccagatcggatgtacctatagtattcggtttcgaggagttagtgtcaccatttctggagggtcgcgaactcttcagcatctcagtgaaatggcaattcggtctaagcaagaactgctacagcttaccccagtcaaagtggaaagtgatgtatcaagaggatgccctgaaggtgttgaaaccttcgaagaagaaagcgagtaaggatcctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcataagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 31 - DNA, 4946 bp, artificial
  T-DNA of pNMD3493
cctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataccatgctagagggcaaagtgaagtggttcaacagcgagaagggattcggctttatcgaagtggaaggccaggatgacgtgtttgttcacttctctgccatccaaggggaaggattcaagacactggaggaaggacaggcagtctcattcgagatagtcgaggggaatagaggacctcaagctgcgaacgttaccaaagaggcttaaggatcctctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacctgcaggcatgccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcagggcccatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctctatttttacaacaattaccaacaacaacaaacaacaaacaacattacaattacatttacaattaccatggaacgagctatacaaggaaacgatgctagggaacaagcttatggtgaacgttggaatggaggatcaggaagttccacttctcccttcaaacttcctgacgaaagtccgagttggactgagtggcggctacataacgatgagacgatttcgaatcaagataatccccttggtttcaaggaaagctggggtttcgggaaagttgtatttaagagatatctcagatacgacgggacggaaacttcactgcacagagtccttggatcttggacgggagattcggttaactatgcagcatctcgatttctcggtttcgaccagatcggatgtacctatagtattcggtttcgaggagttagtgtcaccatttctggagggtcgcgaactcttcagcatctcagtgaaatggcaattcggtctaagcaagaactgctacagcttaccccagtcaaagtggaaagtgatgtatcaagaggatgccctgaaggtgttgaaaccttcgaagaagaaagcgagtaaggatcctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcataagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

(19) United States
(12) Patent Application Publication
(10) Pub. No.: US 2016/0097056 A1
(43) Pub. Date: Apr. 7, 2016

(54) Process of Providing Plants with Abiotic Stress Resistance

(71) Applicant: NOMAD BIOSCIENCE GMBH, Munich (DE)

(72) Inventors: Anka Tümmler, Halle (DE); Doreen Bartels, Köthen (DE); Anatoli Giritch, Halle (DE); Yuri Gleba, Berlin (DE)

(21) Appl. No.: 14/892,866

(22) PCT Filed: May 23, 2014
(86) PCT No.: PCT/EP2014/001403

§ 371 Date: Nov. 20, 2015

(30) Foreign Application Priority Data
May 23, 2013 (EP) 13002691.7

(51) Int. Cl. C12N 15/82

(52) U.S. Cl. CPC C12N 15/8273; C12N 15/8205

(57) ABSTRACT

Crop growth management system comprising a process of providing a plant with improved abiotic stress tolerance, comprising providing aerial parts of said plant with a suspension containing cells of an Agrobacterium strain comprising a nucleic acid molecule comprising a nucleic acid construct containing a nucleotide sequence of interest, said nucleotide sequence of interest providing said plant with increased abiotic stress tolerance upon expression in said plant.

### CROSS REFERENCE TO RELATED APPLICATIONS

This application is the U.S. National Stage of International Application PCT/EP2014/001403, filed May 23, 2014, which designates the U.S. and was published by the International Bureau in English on Nov. 27, 2014, and which claims the benefit of European Patent Application No. 13 002 691.7, filed May 23, 2013; all of which are hereby incorporated herein in their entirety by reference.

### FIELD OF THE INVENTION

The present invention relates to a process of providing a plant with increased resistance against abiotic stress such as drought stress. The invention also relates to an agricultural crop or plant production system and process that allows reacting to abiotic stress occurring during a growing season. The invention also relates to a composition comprising Agrobacterium cells, an Agrobacterium strain and to a suspension containing cells of an Agrobacterium strain for such process. The invention further provides a process of protecting a plurality of plants grown on a farm field from abiotic stress. Further, a plant treated with the composition and having an increased abiotic stress tolerance compared to a plant before treatment is provided.

### BACKGROUND OF THE INVENTION

Abiotic stress tolerance of plants is of great importance for agriculture under harsh environmental conditions, since seed, fruit and biomass production of plants can be impaired substantially by drought or other abiotic stresses that may occur during the vegetation period. Attempts have already been undertaken to increase the tolerance of crop plants to various abiotic stresses (e.g. cold stress, heat stress, water stress, salt stress, drought) by biotechnological means. WO 2005/033318 relates to methods for enhancing stress tolerance in plants, and describes transgenic plants stably transformed with a DNA encoding a cold shock protein from gram positive bacteria, such as Bacillus subtilis. Increased abiotic stress tolerance is reported for transgenic plants. One cold shock protein mentioned in WO 2005/033318 is B. subtilis CspB.

Castiglioni et al., Plant Physiology 147 (2008) 446-455 relates to bacterial RNA chaperones that confer abiotic stress tolerance in plants and improved grain yield in maize under water-limited conditions. Stress tolerance at both vegetative and reproductive stages is reported with enhanced yield stability in maize under water-limiting conditions. Also in this study, plants transgenic for genes encoding cold shock proteins were used.

Abiotic stress is due to environmental conditions and thus generally affects the entire plant. This seems to be the reason as to why transgenic plants have been used in the prior art for providing plants with proteins that increase stress tolerance. However, generation of transgenic crop plant lines is a time-consuming process, requiring many years of development. Moreover, planting of transgenic crops is not allowed or severely restricted (such as to use as animal feed) in several countries, whereby the approaches described in the prior art cannot be used in such countries. Further, transgenic plants generally contain selectable marker genes that were used for selecting transformed plants or cells thereof. Use of such selectable marker genes in transgenic plants is a major concern by critics of transgenic plants, as spread of antibiotic resistance genes may impair the utility of antibiotics in medicine, and spread of herbicide resistance genes to weeds may impair the utility of herbicides. Thus, the abiotic stress protection methods of WO 2005/033318 and Castiglioni et al. are ecologically problematic.

It is an object of the invention to provide a process of providing a plant with increased abiotic stress (notably drought) resistance. It is another object of the invention to provide a process of providing a plant with increased abiotic stress (notably drought) resistance, which is of improved environmental safety and/or does not require spread of antibiotic or herbicide resistance genes in the environment. It is another object of the invention to provide a process of providing crop plants with increased abiotic stress (notably drought) resistance that can be used in countries where planting of transgenic crop plants is not allowed.

### SUMMARY OF THE INVENTION

Accordingly, the present invention provides the following:

(1) A process of providing a plant with improved abiotic stress tolerance, comprising providing aerial parts of said plant with a suspension containing cells of an Agrobacterium strain comprising a nucleic acid molecule comprising a nucleic acid construct containing a nucleotide sequence of interest, said nucleotide sequence of interest providing said plant with increased abiotic stress resistance upon expression in said plant.

(2) A process of providing a plurality of plants with improved abiotic stress tolerance, comprising
growing said plurality of plants on a farm field;
determining whether an abiotic stress affects said plants;
providing aerial parts of said plants with a suspension containing cells of an Agrobacterium strain comprising a nucleic acid molecule comprising a nucleic acid construct containing a nucleotide sequence of interest, said nucleotide sequence of interest providing said plants with increased abiotic stress resistance upon expression in said plants.

(³) The process of providing a plant with improved abiotic stress tolerance according to item (1), comprising
growing said plant up to a desired growth state; and
expressing, in said plant, said nucleotide sequence of interest providing said plant with increased abiotic stress tolerance, comprising providing aerial parts of said plant with said suspension containing cells of said Agrobacterium strain.

(4) The process according to any one of items 1 to 3, wherein, before said step of providing aerial parts of said plant with said suspension, said plant or said plurality of plants does not contain a gene that provides said plant(s) with said abiotic stress tolerance.

(5) The process according to any one of items 1 to 4, wherein said abiotic stress tolerance is drought tolerance.

(6) The process according to any one of items 1 to 5, wherein said nucleotide sequence of interest encodes a protein capable of providing said plant(s) with said increased abiotic stress tolerance.

(7) The process according to item 6, wherein said protein
comprises or consists of an amino acid sequence of any one of the amino acid sequences of SEQ ID NO: 2, 4, 6, 8, 10, 12, 14, 16, 18, 20, 22, and 24; or
comprises or consists of an amino acid sequence having at least 80% sequence identity to the entire amino acid sequence of any one of SEQ ID NO: 2, 4, 6, 8, 10, 12, 14, 16, 18, 20, 22, and 24; or
comprises or consists of an amino acid sequence of a length of at least 80%, preferably at least 90%, more preferably at least 95% of the number of amino acid residues of any one of SEQ ID NO: 2, 4, 6, 8, 10, 12, 14, 16, 18, 20, 22, and 24 and a sequence identity of at least 90% to SEQ ID NO: 2, 4, 6, 8, 10, 12, 14, 16, 18, 20, 22, and 24, respectively, over such length; or
comprises or consists of an amino acid sequence having from 1 to 20 amino acid additions, substitutions or deletions compared to the amino acid sequence of any one of SEQ ID NO: 2, 4, 6, 8, 10, 12, 14, 16, 18, 20, 22, and 24.

(8) The process according to item 6, wherein said protein has a cold shock domain, preferably said protein is an RNA chaperone having a binding site for single-stranded RNA.

(9) The process according to any one of items 6 to 8, wherein said protein comprises the amino acid sequence of SEQ ID NO: 2 or of SEQ ID NO: 4, or said protein comprises an amino acid sequence having at least 80% identity over the entire range of SEQ ID NO: 1 or NO:2.

(10) The process according to item 9, wherein said protein having at least 80% identity over the entire range of SEQ ID NO:2 has at least the amino acid residues of CspA from E. coli W11, F18, F20, F31, H33, and F34 at positions corresponding to these positions in CspA from E. coli.

(11) The process according to any one of items 6 to 10, wherein said protein is CspB from B. subtilis.

(12) The process according to any one of items 1 to 11, wherein said suspension that may be aqueous contains said cells of said Agrobacterium strain in a concentration of at most 2.2.107, preferably of at most 1.1.107, more preferably of at most 4.4.106, more preferably of at most 1.1.106 cfu/ml of said suspension.

(13) The process according to any one of items 1 to 12, wherein said suspension further contains and abrasive suspended in said suspension, said abrasive preferably being a particulate inorganic carrier for wettable powders, such as silica or carborundum.

(14) The process according to item 13, wherein said suspension contains said abrasive in an amount of between 0.02 and 2, preferably between 0.05 and 1 and more preferably between 0.1 and 0.5% by weight of said suspension.

(15) The process according to any one of items 1 to 14, wherein said suspension further comprises an agricultural spray adjuvant, preferably a non-ionic surfactant or wetting agent, preferably the spray adjuvant is an organo-silicone wetting agent, such as Silwet L-77.

(16) The process according to any one of items 1 to 15, wherein said nucleic acid construct is flanked by a T-DNA border sequence on at least one side, which permits the transfer of said nucleic acid construct into cells of said plant.

(17) The process according to any one of items 1 to 16, wherein said nucleic acid construct is present in T-DNA and flanked both sides by T-DNA border sequences, said T-DNA not containing a selectable marker allowing selection of plant or plant cell containing said T-DNA.

(18) The process according to any one of items 1 to 17, wherein said nucleotide sequence of interest is operably linked to a promoter active in plant cells.

(19) The process according to any one of items 1 to 18, wherein said nucleic acid construct encodes a DNA or RNA replicon encoding said protein.

(20) DNA molecule or vector for Agrobacterium-mediated transformation of plants, comprising in T-DNA a nucleic acid construct containing a DNA sequence of interest operably linked to a promoter; said DNA sequence of interest encoding a protein capable of providing a plant with increased abiotic stress tolerance; said T-DNA not containing a selectable marker allowing selection of plant cells containing said T-DNA.

(21) Agrobacterium cell comprising the DNA molecule of item 20.

(22) Agrobacterium strain comprising a heterologous DNA molecule comprising in T-DNA a nucleic acid construct containing a heterologous DNA sequence of interest operably linked to a promoter; said heterologous DNA sequence of interest encoding a protein capable of providing a plant with increased drought resistance; said T-DNA not containing a selectable marker allowing selection of plant cells containing said T-DNA.

(23) Composition containing cells of an Agrobacterium strain, said Agrobacterium strain comprising a heterologous DNA molecule comprising a nucleic acid construct containing a heterologous DNA sequence of interest operably linked to a promoter; said nucleotide sequence of interest providing a plant with increased abiotic stress resistance upon expression in said plant; said composition optionally further comprising a preferably non-ionic wetting agent such as an organosilicone surfactant.

(24) The composition according to item 23, further comprising at least one abrasive.

(25) The composition according to item 23 or 24, wherein said composition is a suspension containing cells of said Agrobacterium strain and at least one abrasive suspended in said suspension, wherein said suspension contains said cells of said Agrobacterium strain in a concentration of at most 4.4.107, preferably of at most 1.1.107, preferably of at most 4.4.106, more preferably of at most 1.1.106 cfu/ml of said suspension.

(27) A plant treated with a composition containing cells of an Agrobacterium strain comprising a nucleic acid molecule comprising a nucleic acid construct containing a nucleotide sequence of interest, said nucleotide sequence of interest providing said plant with increased abiotic stress resistance upon expression in said plant; said plant containing in cells thereof said nucleotide sequence of interest and expresses said nucleotide sequence of interest.

(28) Use of the DNA molecule of item 20, the Agrobacterium cell of item 21, the Agrobacterium strain of item 22 or the composition of any one of items 23 to 25 for conferring abiotic stress tolerance to a plant.

(29) Method of conferring abiotic stress tolerance to a plant, comprising the following steps: preparing a suspension containing cells of an Agrobacterium strain comprising a nucleic acid molecule comprising a nucleic acid construct containing a nucleotide sequence of interest, said nucleotide sequence of interest providing said plant with increased abiotic stress resistance upon expression in said plant; and applying the suspension prepared in the previous step to aerial parts of said plant.

The inventors have surprisingly found that it is possible to achieve increased abiotic stress resistance such as drought resistance in plants by transiently expressing in plants endangered by an abiotic stress a nucleotide sequence of interest providing said plant with increased abiotic stress resistance upon expression in said plant. In one embodiment, the nucleotide sequence of interest encodes a protein capable of providing said plants with increased abiotic stress resistance. The invention provides an abiotic stress protection system, kit and method for plants, wherein provision of a foreign gene (such as the nucleotide sequence of interest) to said plant can be restricted to environmental conditions where abiotic stress such as drought occurs or is expected to occur. If no abiotic stress occurs or is to be expected, providing a foreign gene or transgene e.g. encoding said protein to the plants is not necessary and can be avoided. Consequently, the process of the invention is useful even for countries where agriculture employing transgenic plants (containing a foreign gene stably and heritably incorporated into a chromosome) is not allowed.

The invention provides and enables a crop management system wherein plants that were provided with increased abiotic stress resistance according to the processes of the invention may be excluded from being used for food production for humans, but may instead be used for animal feed production or for biofuel production. However, plants that were not provided with increased abiotic stress resistance due to favourable environmental conditions in a growing season may be used for human food production. The invention represents a large improvement compared to prior art abiotic stress improvement methods (WO 2005/033318; Castiglioni et al., Plant Physiology 147 (2008) 446-455) wherein transgenic plants are planted even in growing seasons where no improved stress tolerance is needed.

Since the nucleotide sequence of interest that is, upon expression, capable of providing said plants with increased abiotic stress resistance is provided to said plants transiently, i.e. without the need for a selectable marker gene such as an antibiotic resistance gene or a herbicide resistance gene and without selecting for plant cells having incorporated such antibiotic resistance gene or a herbicide resistance gene, no antibiotic resistance gene or a herbicide resistance gene is inserted into plants, and spread of such genes in the environment with these plants can be largely avoided. Thus, also for this reason, the process of the invention has improved environmental safety compared to the abiotic stress protection systems of the prior art that rely on transgenic plants. Providing the nucleotide sequence of interest of the invention transiently to plants under abiotic stress conditions provides a selective advantage for the treated plants.

### BRIEF DESCRIPTION OF THE FIGURES

FIG. 1 shows schematically plasmid vectors used for cloning of genes of interest. pNMD2492 is a transcriptional non-replicating vector. RB and LB stand for the right and left borders of T-DNA. P35S: cauliflower mosaic virus 35S promoter; 0: omega translational enhancer; P19: PTGS suppressor P19 from Tomato Bushy Stunt Virus; Tnos: nopaline synthase terminator; Tocs: ocs terminator. pNMD035 is a TMV-based viral vector with cell-to cell movement ability. Pact2: promoter of Arabidopsis actin2 gene; o: 5′ end from TVCV (turnip vein clearing virus); RdRp: RNA-dependent RNA polymerase open reading frame (ORF) from cr-TMV (crucifer-infecting tobamovirus); MP: movement protein ORF from cr-TMV; N: 3′-non-translated region from cr-TMV; Tnos or nos: nopaline synthase terminator; white segments interrupting grey segments in the RdRp and MMP ORFs indicate introns inserted into these ORFs for increasing the likelihood of RNA replicon formation in the cytoplasm of plant cells, which is described in detail in W02005049839. pNMD661 is a TMV-based vector lacking cell-to cell movement ability. A point mutation in the MP ORF leads to a frame shift preventing correct MP translation. pNMD670 is a PVX (potato virus X)-based vectors with cell-to-cell movement ability. PVX-pol: RNA-dependent RNA polymerase from PVX; CP: coat protein ORF; 25K, 12K and 8 together indicate the 25KDA, 12 kDa and 8 kDa triple gene block modules from PVX; N: 3′-untranslated region from PVX. pNMD694 is a PVX-based vector with deletion of the coat protein coding sequence were disabled for both systemic and cell-to cell movement.

FIG. 2 shows schematically transcriptional vectors for the expression of Arabidopsis thaliana nuclear transcription factor Y subunit B-1 (NF-YB1) (GenBank: NM_129445.2) (plasmid construct pNMD3486) and Cold shock protein CspB from Bacillus subtilis subsp. subtilis str JH642 (GenBank: AAB01346) (plasmid construct pNMD3493).

FIG. 3 shows Brassica napus N90-740 plants 11 days post spraying, 11 days drought.

FIG. 4 shows Brassica napus N90-740 plants 18 days post spraying, 11 days drought, 7 days rewatering.

FIG. 5 shows Brassica napus N90-740 plants 26 days post spraying, 11 days drought, 7 days rewatering, 8 days drought.

FIG. 6 shows Brassica napus N90-740 plants 26 days post spraying, 11 days drought, 7 days rewatering, 13 days drought, 1 day rewatering.

FIG. 7 shows schematically PVX viral vectors with systemic movement ability used for expression of genes of interest in tomato plants. RB and LB stand for the right and left borders of T-DNA. P35S: cauliflower mosaic virus 35S promoter; PVX-POL: RNA-dependent RNA polymerase from PVX; CP: coat protein ORF; 25K, 12K and 8 together indicate the 25KDA, 12 kDa and 8 kDa triple gene block modules from PVX; N: 3′-untranslated region from PVX; GmRD22: coding sequence of GmRD22 (apoplast-localized BURP-domain protein) from soybean Glycine max; BnLAS: coding sequence of BnLAS (GRAS family gene) from rapeseed Brassica napus; GFP: coding sequence of Green Fluorescent Protein from jellyfish. VirG N54D stands for the coding sequence of VirG protein from Agrobacterium tumefaciens GV3101 with N54D mutation.

FIG. 8 shows phenotypes of transfected tomato Lycopersicon esculentum cv. Balcony Red plants exposed to drought three weeks post spraying with agrobacteria harboring PVX vectors for the expression of BnLAS and GmRD22 genes. Drought exposure was performed for 14 days.

FIG. 9 shows fruits of transfected tomato Lycopersicon esculentum cv. Balcony Red plants exposed to drought three weeks post spraying with agrobacteria harboring PVX vectors for the expression of BnLAS and GmRD22 genes. Drought exposure was performed for 14 days. Fruit yield was evaluated 100 days post spraying. Numerals 1, 2, 3 etc. stand for individual plants.

FIG. 10 shows biomass of fruits harvested from transfected tomato Lycopersicon esculentum cv. Balcony Red plants exposed to drought three weeks post spraying with agrobacteria harboring PVX vectors. Drought exposure was performed for 14 days. Fruit yield was evaluated 100 days post spraying and calculated as an average per plant. 1: untransfected plant; 2: plants expressing BnLAS gene; 3: plants expressing GmRD22 gene; 4: plants expressing GFP.

FIG. 11 depicts phenotypes of GmRD22, BnLAS and GFP transfected tomato Balcony Red seedlings exposed to the PEG6000 treatment.

FIG. 12 shows Lycopersicon esculentum cv. Balcony Red seedlings transfected with GFP and BnLAS expressing PVX vectors after 10 days of withholding irrigation and following recovery (2h after watering). Plants were sprayed with agrobacteria 12 days post sowing and exposed to drought 12 days post spraying.

### DETAILED DESCRIPTION OF THE INVENTION

The processes of the invention have their main application in agriculture, i.e. on a large scale. Thus, these processes are generally carried with a plurality of plants. Accordingly, the processes are generally applied to a plurality of plants sown or planted e.g. on a farm field. Before carrying out the process of the invention, the sown or planted plants preferably do not contain an expressible gene that provides the plants with the abiotic stress tolerance provided in the processes of the invention. Notably, the plants are preferably not transgenic for such gene.

After having sown or planted the plants, a user which is typically a farmer may observe growth of the plants and the health state of the plants. Further, the user may observe environmental conditions such as the weather and/or the condition of the soil for determining whether abiotic stress conditions for the plants have developed, are developing or may develop. Determining whether abiotic stress conditions are present may involve taking samples from the soil and examining the soil. Alternatively or additionally, determining whether abiotic stress conditions are present may involve taking samples from the plants or from a fraction of said plants and examining the plants.

For determining drought stress or salt stress, one ore more soil samples may be examined for water content or salt content, respectively. In a preferred embodiment, the processes of the invention are used for improving the tolerance or resistance (these terms are used interchangeably herein) of the plants against drought. Thus, determining drought stress and the further development of such stress may involve following weather forecasts for assessing the probability of rain.

Determining drought stress conditions is known in the art, see e.g. Jones et al., Journal of Experimental Botany 58 (No. 2) 119-130, 2007; Idso, Agricultural Meteorology 27, 59-70, 1982; Shimada et al., Journal of Arid Land Studies 22-1, 251-254, 2012. Soil measures and/or plant measures may be used for determining or following a water status affecting the plants. The known methods may also be classified into direct methods and indirect methods. Among direct methods, measurement of the relative water content (RWC) of soil or plant material and direct measurement of the leaf water potential e.g. with a pressure chamber, or using of sap flow sensors may be mentioned. Among indirect methods, remote infrared technologies based on the evaluation of canopy temperature or remote spectrometric methods based on the measuring of canopy spectral reflectance may be mentioned. As water becomes limiting, crop temperatures rise because they cannot transpire enough water to keep themselves cool. Plant leaves open their stomata to admit carbon dioxide for photosynthesis and at the same time water vapor flows out of the leaf, which cools the leaf surface. When soil water becomes limiting, transpiration decreases, thus reducing the leaf cooling effect and causing the crop temperature to rise. All objects emit energy or radiation that is measured as their temperature. For crop canopies the temperature is usually measured with a thermal infrared thermometer. Infrared thermometers (IRT) are commercially available. Irmak et al., Agronomy Journal, 92, 1221-1227, 2000) describe the determination of crop water stress index (CWSI) for the purpose of irrigation timing, but may also be used for determining drought stress in plants for the present invention. Other measures for following the water status are turgor pressure or stromatal conduct, and water potential in soil or the plants. In order not to depend on absolute determinations of the water status measures mentioned above, one may follow the water status continuously or regularly for easily recognizing development of drought stress.

At a desired growth state of the plant(s) or when the user has determined that resistance of the plants against an abiotic stress should be improved, the plants or parts thereof are provided with a suspension containing cells of an Agrobacterium strain comprising a nucleic acid molecule comprising a nucleic acid construct containing a nucleotide sequence of interest. The nucleotide sequence of interest can, upon expression in the plants, provide the plants with increased tolerance against the abiotic stress.

The invention also provides a process of producing plants or parts of said plants, comprising the following steps:
(A) growing a plurality of plants on a field;
(B) determining whether drought stress affects said plants;
(C) if it was determined in step (B) that drought stress affects or may affect said plants, providing aerial parts of said plants with a suspension containing cells of an Agrobacterium strain comprising a nucleic acid molecule comprising a nucleic acid construct containing a nucleotide sequence of interest, said nucleotide sequence of interest providing said plants with increased drought stress resistance upon expression in said plants;
(D) harvesting said plants or desired parts thereof; and
(E) if step (C) was performed, using the harvest of step (D) for a purpose for which having performed step (C) is acceptable; or, if step (C) was not performed, using the harvest of step (D) for a purpose for which having performed step (C) is not acceptable..

A purpose for which performing step (C) may not be acceptable is food production for human consumption. A purpose for which performing step (C) may be acceptable is animal feed production or production of biofuel. Thus, the invention provides a crop production system and method for entire growing seasons, that strongly improves overall efficiency. On the one hand, loss of crop or plant material due to drought can be reduced, and the saved crops or biomass can be used for purposes for which genetic modification is not of concern. On the other hand, in seasons where no measures for increasing the drought tolerance are required due to favourable weather conditions, the plants grown are non-genetically modified, which is preferred by some consumers or in some countries.

In the present invention, the nucleotide sequence of interest may encode a protein capable of providing said plants with increased abiotic stress resistance such as drought resistance. Alternatively, the nucleotide sequence of interest may produce, upon expression, an RNA that may provide the plants with the increased abiotic stress resistance.

The nucleic acid molecule is generally a DNA molecule. In this case, the nucleotide sequence of interest contained therein is DNA sequence of interest.

In the processes of the invention, parts of said plants are transiently transfected with said nucleic acid molecule (vector) for transient expression of said nucleotide sequence of interest. This has the advantage that a decision on when the nucleotide sequence of interest should be expressed in plants, and which nucleotide sequence of interest to be expressed, can be delayed until shortly before the point of transfection. The term “transient” means that no selection methods are used for selecting cells or plants transfected with the nucleic acid molecule (vector) or with the nucleic acid construct from non-transfected cells or plants using, e.g. a selectable agent and a selectable marker gene capable of detoxifying the selectable agent. As a result, the transfected nucleic acid is generally not stably introduced into plant chromosomal DNA. Instead, transient expression methods make use of the effect of transfection in the very plant cells transfected.

A possible way of achieving expression of said nucleotide sequence of interest is the use of self-replicating (viral) replicons containing a nucleotide sequence encoding said nucleotide sequence of interest. Plant viral expression systems have been described in many publications both for the use of DNA viral vectors and for the use of RNA viral vectors, such as in WO2008028661, WO2006003018, WO2005071090, WO2005049839, WO2006012906, WO02101006 or WO02068664 and many more publications are cited in these documents.

Various methods for introducing a nucleic acid molecule, such as a DNA molecule, into a plant or plant part for transient expression are known. In the invention, Agrobacteria are preferably used for transfecting plants with the nucleic acid molecule (vector) or nucleic acid construct e.g. by agroinfiltration. A system and method for large scale infiltration of plants by agrobacteria is described in WO2009095183. In another embodiment, plants or plant parts are sprayed with a suspension containing cells of an Agrobacterium strain, which is well suitable for large scale applications to many plants such as to plants on a farm field. Such spray transfection processes are described in detail in WO2012/019660.

The Agrobacterium strain may belong to the species Agrobacterium tumefaciens or Agrobacterium rhizogenes that are commonly used for plant transformation and transfection and which are known to the skilled person from general knowledge. The Agrobacterium strain to be used in the processes of the invention comprises a DNA molecule (Ti-plasmid) as said nucleic acid molecule, comprising a nucleic acid construct containing a DNA sequence of interest. The DNA sequence of interest may encode one or more than one protein to be expressed in plants. The nucleic acid construct is typically present in T-DNA of Ti-plasmids for introduction of the nucleic construct into plant cells by the secretory system of the Agrobacterium strain. On at least one side or on both sides, the nucleic acid construct is flanked by a T-DNA border sequence for allowing transfection of said plant(s) and introduction into cells of said plant of said nucleic acid construct. Preferably, said nucleic acid construct is present in T-DNA and flanked on both sides by T-DNA border sequences.

Most preferably, the nucleic acid construct is present in T-DNA of a Ti-plasmid of the Agrobacterium strain. Ti-plasmids may contain a selectable marker outside of said T-DNA for allowing cloning and genetic engineering in bacteria. However, the T-DNA that is transferred into cells of said plant preferably does not contain a selectable marker that would, if present, allow selection of plant or plant cells containing said T-DNA. Examples of selectable marker genes that should, in this embodiment, not be present in T-DNA of the Ti-plasmid are an antibiotic resistance gene or a herbicide resistance gene. Since the processes of the invention use transient transfection and expression of said nucleotide sequence of interest (or said protein), the processes do not comprise a step of selecting for plant cells having incorporated the nucleic acid molecule of the invention by using such antibiotic resistance gene or a herbicide resistance gene. Accordingly, no antibiotic resistance gene or a herbicide resistance gene needs to be incorporated into said plants, whereby the probability of spreading such genes in the environment is low in the processes of the invention.

The nucleic acid construct comprises the nucleotide sequence of interest such that the latter is expressible in plant cells. For this purpose, the nucleotide sequence of interest may be, in said nucleic acid construct, under the control of a promoter active in plant cells. Preferably, the nucleotide sequence of interest encodes a protein capable of providing plants with increased abiotic stress tolerance. Examples of the nucleotide sequence of interest are a DNA sequence encoding a DNA viral replicon or an RNA viral replicon, or a gene to be expressed. The gene provides, upon expression, the plant with the increased abiotic stress tolerance. Preferably, the gene encodes a protein capable of providing the plant with increases abiotic stress tolerance. Also the viral replicons encode such protein to be expressed in cells of the plant(s). The nucleic acid construct may comprise, in addition to the nucleotide sequence of interest, other sequences such as regulatory sequences for expression of the nucleotide sequence of interest, such as a transcription promoter and terminator. The nucleic acid construct may comprise a further gene to be expressed, e.g. a gene encoding a suppressor of gene silencing such as the P19 protein. Expression of such further gene may be under the control of the same or a different promoter as the promoter used for expressing the protein of the invention. Agrobacterium-mediated gene transfer and vectors therefor are known to the skilled person, e.g. from the references cited above or from text books on plant biotechnology such as Slater, Scott and Fowler, Plant Biotechnology, second edition, Oxford University Press, 2008.

As used herein, the term “promoter active in plant cells” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the cauliflower mosaic virus 35S promoter (CaMV35S promoter) (Harpster et al. (1988) Mol Gen Genet. 212(1):182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters (such as the light-inducible Rubisco promoters), root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like. For transient expression, constitutive promoters, i.e. promoters that are not developmentally regulated, are preferably used. However, constitutive promoters may be tissue-specific or organ-specific. Preferred promoters are those used in the Examples described below.

Herein, the term “construct” means a recombinant construct comprising a nucleotide sequence of interest. Preferably, the construct encodes at least a protein capable of providing said plants with increased abiotic stress resistance.

In embodiments wherein strong expression of the protein is desired, the nucleic acid construct may encode a viral vector that can replicate in plant cells to form replicons of the viral vector. In order to be replicating, the viral vector and the replicons contain an origin of replication that can be recognized by a nucleic acid polymerase present in plant cells, such as by the viral polymerase expressed from the replicon. In case of RNA viral vectors (referred to as “RNA replicons”), the replicons may be formed by transcription under the control of a promoter active in plant cells, from the DNA construct after the latter has been introduced into plant cell nuclei. In case of DNA replicons, the replicons may be formed by recombination between two recombination sites flanking the sequence encoding the viral relicon in the DNA construct, e.g. as described in WO00/17365 and WO 99/22003. If the replicon is encoded by the DNA construct, RNA replicons are preferred. Use of DNA and RNA viral vectors (DNA or RNA replicons) has been extensively described in the literature over the years. Some examples are the following patent publications: WO2008028661, WO2007137788, WO 2006003018, WO2005071090, WO2005049839, WO02097080, WO02088369, WO02068664. An example of DNA viral vectors are those based on geminiviruses. For the present invention, viral vectors or replicons based on plant RNA viruses, notably those based on plus-sense single-stranded RNA viruses may be used. Accordingly, the viral replicon may be a plus-sense single-stranded RNA replicon. Examples of such viral vectors those based on tobacco mosaic virus (TMV) and potex virus X (PVX). “Based on” means that the viral vector uses the replication system such as the replicase and/or other proteins involved in replication of these viruses. Potexvirus-based viral vectors and expression systems are described in EP2061890 or WO2008/028661. Many other plant viral replicons are described in the patent publications mentioned above.

In one embodiment, the processes of the invention comprise spraying aerial parts of said plant with a suspension containing cells of an Agrobacterium strain comprising a DNA molecule comprising a nucleic acid construct containing a DNA sequence of interest preferably encoding said protein.

The suspension that may be used for providing aerial parts of said plant is a liquid composition comprising Agrobacterium cells suspended in the liquid. The suspension is generally aqueous, which means that the liquid contains at least 50, preferably at least 80, more preferably at least 90% by weight water. Other components in the liquid may be other liquid components compatible with the maintenance of the Agrobacterium cells in a viable form, which may be polar solvents such as polyhydric alcohols or dimethylsulfoxide. Further components may be nutrients, minerals or salts, buffering agents or spray adjuvants (see further below).

The suspension used for spraying plants or plant parts in the process of the invention may have a concentration of Agrobacterium cells of at most 1.1.10⁹ cfu/ml, which corresponds approximately to an Agrobacterium culture in LB-medium of an optical density at 600 nm of 1. Due to the high transfection efficiency achievable, notably if spraying is done with the use of an abrasive and/or a surfactant or wetting agent (see below), much lower concentrations may, however, be used, which allows treatment of many plants such as entire farm fields without the need for huge fermenters for Agrobacterium production. Thus, the concentration may be at most 2.2.10⁷ cfu/ml, preferably at most 1.1.10⁷ cfu/ml, more preferably at most 4.4.10⁶ cfu/ml. In one embodiment, the concentration is at most 1.1.10⁶ cfu/ml of the suspension. For avoiding determination of cell concentrations in terms of cfu/ml, concentrations of agrobacterial suspensions are frequently assessed by measuring the apparent optical density at 600 nm using a spectrophotometer. Herein, the concentration of 1.1.10⁷ cfu/ml corresponds to a calculated optical density at 600 nm of 0.01, whereby the calculated optical density is defined by a 100-fold dilution with water or buffer of a suspension having an optical density of 1.0 at 600 nm. Similarly, the concentrations of 4.4.10⁶ cfu/ml and 1.1.10⁶ cfu/ml correspond to a calculated optical density at 600 nm of 0.004 and 0.001, respectively, whereby the calculated optical densities are defined by a 250-fold or 1000-fold, respectively, dilution with water or buffer of a suspension having an optical density of 1.0 at 600 nm.

The suspension containing cells of an Agrobacterium strain used for spraying the plants or parts of a plant may contain at least one abrasive suspended in said suspension for improving transfection efficiency. The abrasive that may be used is a particulate material that is essentially insoluble in the aqueous suspension of Agrobacterium cells. The abrasive is believed to weaken, notably if used together with a wetting agent, the surface of plant tissue such as leaves, and thereby facilitates penetration of Agrobacterium cells into the intercellular space of plant tissue. As a result, the transfection efficiency is high.

The particulate material to be used as the abrasive of the invention may be carrier material as commonly used as carriers in wettable powder (WP) of pesticide formulations. In the context of wettable powders, these carriers are also referred to in the field of pesticide formulations as “fillers” or “inert fillers”. Wettable powder formulations are part of the general knowledge in the field of plant protection. Reference is made to the handbook PESTICIDE SPECIFICATIONS, “Manual for Development and Use of FAO and WHO Specifications for Pesticides”, edited by the World Health Organisation (WHO) and the FOOD and Agriculture Organization of the United States, Rome, 2002, ISBN 92-5-104857-6. Wettable powder formulations for plant protection are for example described in EP 1810569, EP1488697, EP1908348 and EP0789510. The abrasive may be a mineral material, typically an inorganic material. Examples of such carrier materials are diatomaceous earth, talc, clay, calcium carbonate, bentonite, acid clay, attapulgite, zeolite, sericite, sepiolite or calcium silicate. It is also possible to use quartz powder such as the highly pure quartz powder described in WO02/087324. Preferred examples are silica, such as precipitated and fumed hydrophilic silica, and carborundum. The abrasive properties of diluents or fillers such as silica used in wettable powders are known (see “Pesticide Application Methods” by G. A. Matthews, third edition, Blackwell Science, 2000, on page 52 thereof).

As commercial products of particulate inorganic materials for use as abrasives, the hydrophilic silica Sipernat™ 22S and Sipernat™ 50 S, manufactured by Evonic Degussa may be mentioned. Other products are “Hi-Sil™ 257”, a synthetic, amorphous, hydrated silica produced by PPG Industries Taiwan Ltd. or “Hubersorb 600™”, a synthetic calcium silicate, manufactured by Huber Corporation. A commercial sub-micron sized silica is Hi-SiI™ 233 (PPG Industries) having an average particle size of around 0.02 μm.

The abrasive may have a median particle size between 0.01 and 40, preferably between 0.015 and 30, more preferably between 0.05 and 30, even more preferably between 0.1 and 30, even more preferably between 0.1 and 20, even more preferably between 0.5 and 20, and most preferably between 1.0 and 16 μm. In one embodiment, the median particle size is between 0.015 and 1 or between 0.02 and 0.5 μm. The median particle size is the volume median particle size that can be measured by laser diffraction using a Mastersizer™ from Malvern Instruments, Ltd. In order to avoid clogging of spraying nozzles, the maximum particle size of the largest particles contained in the abrasive should be at most 45 μm, preferably at most 40 μm, which may be determined by sieving. This condition is considered fulfilled, if the sieve residue is below 1.5% by weight (following ISO 3262-19). The abrasive may have a D90 value of at most 40 μm, preferably of at most 30 μm, measured by laser diffraction as described above. Typically, the particle sizes above relate to primary particle sizes. The content of the abrasive in the aqueous suspension of the invention may be between 0.01 and 3, preferably between 0.02 and 2, more preferably between 0.05 and 1 and even more preferably between 0.1 and 0.5% by weight of said suspension.

The suspension usable for spraying parts of the plants preferably contains an agricultural spray adjuvant. The spray adjuvant may be a surfactant or wetting agent. The surfactant or wetting agent has multiple advantages in the present invention. It reduces the surface tension of the water of the aqueous suspension and makes the waxy surface of plant leaves more permeable for agrobacteria. It further improves the stability of the suspension and reduces settling of the abrasive in the suspension. Surfactants used in the present invention are not particularly limited. However, nonionic surfactants are preferred. A measurement frequently used to describe surfactants is the HLB (hydrophilic/lipophilic balance). The HLB describes the ability of the surfactant to associate with hydrophilic and lipophilic compounds. Surfactants with a high HLB balance associate better with water soluble compounds than with oil soluble compounds. Herein, the HLB value should be 12 or greater, preferably at least 13. As noninionic surfactants, organo-silicone surfactants such as polyalkyleneoxide-modified heptamethyltrisiloxane are most preferred in the present invention. A commercial product is Silwet L77™ spray adjuvant from GE Advanced Materials.

Agrobacterium strains usable in the invention are those that are generally used in the art for transfecting or transforming plants. Generally, binary vector systems and binary strains are used, i.e. the vir genes required for transfer of T-DNA into plant cells on the one hand and the T-DNA on the other hand are on separate plasmids. Examples of usable Agrobacterium strains are given in the article of Hellens et al., Trends in Plant Science 5 (2000) 446-451 on binary Agrobacterium strains and vector systems. In the context of a binary Agrobacterium strain, the plasmid containing the vir genes is referred to as “vir plasmid” or “vir helper plasmid”. The plasmid containing the T-DNA to be transfected is the so-called binary vector that is also referred to herein as “DNA molecule” or “vector”. The term “strain” or “Agrobacterium strain” relates to components of the Agrobacterium other than the binary vector. Thus, herein, a binary Agrobacterium strain not containing a binary vector and after introduction of a binary vector are referred to by the same strain name.

The suspension of agrobacteria may be produced as follows. The DNA molecule or vector containing the nucleic acid construct may be transformed into the Agrobacterium strain and transformed Agrobacterium cultures are grown optionally under application of selective pressure for maintenance of said DNA molecule. In one method, the Agrobacterium strain to be used in the processes of the invention is then inoculated into a culture medium and grown to a high cell concentration. Larger cultures may be inoculated with small volumes of a highly concentrated culture medium for obtaining large amounts of the culture medium. Agrobacteria are generally grown up to a cell concentration corresponding to an OD at 600 nm of at least 1, typically of about 1.5. Such highly concentrated agrobacterial suspensions are then diluted to achieve the desired cell concentration. For diluting the highly concentrated agrobacterial suspensions, water is used. The water may contain a buffer or salts. The water may further contain the surfactant mentioned above. Alternatively, the concentrated agrobacterial suspensions may be diluted with water, and any additives such as the surfactant and the optional buffer substances are added after or during the dilution process. The abrasive may be added before, during or after dilution. It is however preferred to agitate the suspension during addition of the abrasive to uniformly disperse the abrasive in the agrobacterial suspension. The step of diluting the concentrated agrobacterial suspension may be carried out in the spray tank of the sprayer used for spraying the diluted suspensions.

The sprayer to be used for transfecting plants or plant parts in the process of the invention mainly depends on the number of plants or the area to be sprayed. For one or a small number of plants to be sprayed, pump sprayers as widely used in household and gardening can be used. These may have volumes of the spray tank of between 0.5 and 2 liters. For applications on a medium scale, manually operated hydraulic sprayers such as lever-operated knapsack sprayers or manually operated compression sprayers may be used. However, the high transfection efficiency achieved in the invention has its full potential in the transfection of many plants such as plants growing on a farm field or in a greenhouse. For this purpose, power-operated hydraulic sprayers such as tractor-mounted hydraulic sprayers equipped with spray booms can be used. Aerial application techniques using helicopters or airplanes are also possible for large fields. All these types of sprayers are known in the art and are described for example in the book “Pesticide Application Methods” by G. A. Matthews, third edition, Blackwell Science, 2000. In order to ensure a homogeneous suspension in the spray tanks of the sprayers, small or medium size sprayers may be shaken at regular intervals or continuously during spraying. Large sprayers such as the tractor-mounted sprayers should be equipped with an agitator in the spray tank.

Considering the presence of agrobacterial cells and optionally abrasive in the suspensions to be sprayed, sprayers used in the invention should produce spray of a droplet size at least of fine spray. Also, medium spray or coarse spray in the classification of sprays used in the above-mentioned book by G. A. Matthews, page 74, may be used. The main purpose of the spraying in the invention is wetting of plant tissue with the suspension. Thus, the exact droplet size is not critical. However, the transfection efficiency may be further improved by providing the spray to plant surfaces with increased pressure.

It is possible to apply the processes of the invention to transgenic plants containing a gene providing the plant with any desired trait other than the improved abiotic stress resistance trait provided in the processes of the present invention.

The invention also provides a composition containing cells of an Agrobacterium strain, said Agrobacterium strain comprising a heterologous DNA molecule comprising a nucleic acid construct containing a heterologous DNA sequence of interest operably linked to a promoter; said nucleotide sequence of interest providing a plant with increased abiotic stress resistance upon expression in said plant. The composition may be liquid or solid. Examples of solid compositions are dried or lyophilized compositions containing cells of said Agrobacterium strain. An example of a liquid composition is the suspension mentioned below. Lyophilized compositions may be prepared by freeze drying suspensions containing Agrobacterium cells, preferably in the presence of freeze-protection agents such as glycerol. The composition may further comprise a the spray adjuvant or the preferably non-ionic wetting agent such as an organosilicone surfactant mentioned above. The composition may be used for producing the suspension for spraying e.g. by dilution with water or an aqueous solvent.

In the process of the invention, said nucleotide sequence of interest or said protein capable of providing said plants with increased abiotic stress resistance may be expressed in multi-cellular plants, notably, higher plants. Crop plants used in agriculture are preferred. Both monocot and dicot (crop) plants can be used. Common crop plants for the use in present invention include alfalfa, barley, beans, canola, cowpeas, cotton, corn, clover, lotus, lentils, lupine, millet, oats, peas, peanuts, rice, rye, sweet clover, sunflower, sweetpea, soybean, sorghum triticale, yam beans, velvet beans, vetch, wheat, wisteria, and nut plants. The plant species preferred for practicing this invention include, but not restricted to, representatives of Gramineae, Compositeae, Solanaceae and Rosaceae. Preferred plants are plants that do not normally enter the food chain such as Nicotiana species such as N. tabacum and N. benthamiana may be used.

Generally, the protein is expressed in the cytosol of cells of said plants or plant parts. In this case, no signal peptide directing the protein into a particular compartment is added to the enzyme.

The protein capable of providing said plants with increased abiotic stress resistance expressed and used in the present invention is generally a heterologous protein to the plant or plant part in which it is to be expressed in the invention, i.e. the plant or its part does not produce the protein naturally. It is possible to optimise codon usage of genes encoding these enzymes for expression in plants, notably in the plant employed for expressing them. Codon optimisation is a standard method for increasing expression yields. Codon-optimised genes and nucleic acids can be ordered from commercial sources such as Entelechon GmbH, Regensburg, Germany.

Increased abiotic stress resistance of a plant may be determined by treating a plant with the suspension of the invention and treating, as a control or reference, the same type of plant under otherwise identical conditions with a suspension not containing said cells of said Agrobacterium strain. Both plants are then subjected to identical conditions of abiotic stress. Increased abiotic stress resistance of the plant treated according to the invention can be detected by a more vigorous growth or better health condition.

Examples of abiotic stress resistances that may be increased according to the invention are the following: drought resistance, salt resistance, osmotic stress resistance, cold resistance, freezing resistance, heat resistance, resistance to heavy metals, resistance to hypoxia or oxidative stress resistance.

Examples of nucleotide sequences of interest and proteins for improving drought stress tolerance in plants are given in the following. References to the genes and proteins are also given.

CspB from Bacillus subtilis subsp. subtilis. Str, such as JH642 (AAB01346). Castiglioni et al., Plant Physiology 147 (2008) 446-455 and Hunger et al., J. Bacteriol. 188 (2006) 240-248. The coding sequence of CspB codon-optimised for expression in plants is given SEQ ID NO: 1. The amino acid sequence is given as SEQ ID NO: 2.

E.coli cold shock protein 7.4 (cspA) gene. A coding sequence of the CspA protein suitable for expression in plants is given SEQ ID NO: 3. The amino acid sequence is given as SEQ ID NO: 4.

Arabidopsis thaliana nuclear transcription factor Y subunit B-1 (NF-YB1) (NM_129445.2) increases drought resistance in transgenic plants; cf. Nelson et al., PNAS 104 (2007) 16450-16455. A coding sequence is given SEQ ID NO: 5. An amino acid sequence is given as SEQ ID NO: 6.

BnLAS-GRAS family gene. Ectopic expression thereof in plants inhibits growth, delays flowering, delays leaf senescence and increases drought resistance, cf. Yang et al. (2011) Plant Cell Rep 30(3): 373-88. A coding sequence is given SEQ ID NO: 7. An amino acid sequence is given as SEQ ID NO: 8.

IPT (isopentenyl transferase) expression prevents the degradation of photosynthetic protein complexes during drought, cf. Rivero et al., Plant Cell Physiol. 51 (2010) 1929-1941, and Ori et al., The Plant Cell 11(1999) 1073-1080. An IPT coding sequence is given as SEQ ID NO: 21. An amino acid sequence is given as SEQ ID NO: 22.

BcWRKY46-TF, a member of WRKY protein family, was reported to enhance the cold, salt and dehydration stress tolerance in tobacco, cf. Wang et al., Mol. Biol. Rep 39 (2012) 4553-4564. A coding sequence of BcWRKY46 is given SEQ ID NO: 11. An amino acid sequence is given as SEQ ID NO: 12.

HaHB1 from sunflower, AtHB13, AtPR2, AtPR4 and AtGLU-homeodomain-leucine zipper transcription factors confer tolerance to drought and salinity stress, cf. Cabello & Chan (2012) Plant Biotechnol. J. 10 (2012) 815-825. A complete coding sequence with removed splice sites is given SEQ ID NO: 13. An amino acid sequence is given as SEQ ID NO: 14.

Harpins from gram-negative plant pathogenic bacteria—stimulate hypersensitive cell death (HCD), pathogen defense and enhance growth in plants, drought tolerance, cf Zhang et al., J. Exp. Bot. 62 (2011) 4229-4238.

AVP1 H+ Pump (vacuolar H 1-pyrophosphatase) - plants overexpressing the vacuolar H1-pyrophosphatase are much more resistant to high concentrations of NaCI and to water deprivation than the isogenic wild-type strains; cf. Gaxiola et al. 2001 PNAS 98 (20) 11444-11449; Park et al., PNAS 102 (2005) 18830-18835; Zhang et al. (2011) Plant Signaling & Behavior 6: 6, 861-863. A coding sequence is given as SEQ ID NO: 17. An amino acid sequence is given as SEQ ID NO: 18.

Stress responsive gene SNAC1 (STRESS-RESPONSIVE NAC 1) from rice (GenBank: DQ394702)-SNAC1 (STRESS-RESPONSIVE NAC 1) significantly enhances drought resistance in transgenic rice in the field under severe drought stress conditions at the reproductive stage while showing no phenotypic changes or yield penalty, cf. Hu et al. (2006) PNAS 103: 12987-12992. A coding sequence is given as SEQ ID NO: 19. An amino acid sequence is given as SEQ ID NO: 20.

Salt tolerance may be increased by expressing in plants the following proteins:

GmRD22 (apoplast-localized BURP-domain protein from soybean) increases salinity and osmotic stress tolerance in transgenic Arabidopsis and rice, increases lignin production, cf. Wang et al. (2012) Plant Cell Environ. 35 (2012) 1932-1947. A coding sequence of GmRD22 is given SEQ ID NO: 9. An amino acid sequence is given as SEQ ID NO: 10.

HaHB1 from sunflower, AtHB13, AtPR2, AtPR4 and AtGLU-homeodomain-leucine zipper transcription factors as mentioned above can be used for increasing salt tolerance of plants.

Suppressed expression of the apoplastic ascorbate oxidase gene increases salt tolerance in plants, cf. Yamamoto et al. J. Exp Botany 56 (2005) 1785-1796. A coding sequence of the gene from N. tabacum is given SEQ ID NO: 15. An amino acid sequence is given as SEQ ID NO: 16. Expression of ascorbate oxidase may be suppressed by using RNA interference as is known in the art.

AVP1 H+ Pump (vacuolar H 1-pyrophosphatase)—Transgenic plants overexpressing the vacuolar H1-pyrophosphatase are much more resistant to high concentrations of NaCI and to water deprivation than the isogenic wild-type strains; cf. Gaxiola et al. 2001 PNAS 98 (20) 11444-11449; Zhang et al. (2011) Plant Signaling & Behavior 6: 6, 861-863; for sequences see above.

Stress responsive gene SNAC1 (STRESS-RESPONSIVE NAC 1) from rice (GenBank: DQ394702)—SNAC1 (STRESS-RESPONSIVE NAC 1) enhances drought salt tolerance in transgenic rice in the field under severe drought stress conditions, cf. Hu et al. (2006) PNAS 103: 12987-12992. A coding sequence is given as SEQ ID NO: 19. An amino acid sequence is given as SEQ ID NO: 20.

Cold tolerance may be increased by expressing in plants the following protein:

BcWRKY46-TF, the member of WRKY protein family. Constitutive expression in transgenic tobacco reduced susceptibility to cold, ABA, salt and dehydration stress. Wang et al. (2012) Mol Biol Rep.

The SNAC1-targeted gene OsSRO1c modulates oxidative stress tolerance by regulating hydrogen peroxide in rice; cf. You et al., J. Experimantal Botany (2013).

LeCYP1-Tomato Cyclophilin. A coding sequence is given as SEQ ID NO: 23. An amino acid sequence is given as SEQ ID NO: 24. Drought resistance though improved root formation;cf. Oh et al. (2006) Planta 224: 133-144.

In order to provide plants with increased abiotic stress resistance according to the present invention, any of the proteins of SEQ ID NO: 2, 4, 6, 8, 10, 12, 14, 16, 18, 20, 22 and 24 may be expressed in the plants. The same aim may be achieved by expressing in said plants a protein comprising an amino acid sequence having a sequence identity to any of the sequences of SEQ ID NO: 2, 4, 6, 8, 10, 12, 14, 16, 18, 20, 22, and 24 of less than 100%. The protein to be expressed may be a variant protein having an amino acid sequence having at least 80% sequence identity to the entire amino acid sequence of any one of SEQ ID NO: 2, 4, 6, 8, 10, 12, 14, 16, 18, 20, 22, and 24. The amino acid sequence identity may be at least 85%, preferably at least 90%, more preferably at least 95% and even more preferably at least 97% to any one of amino acid sequence of any one of SEQ ID NO: 2, 4, 6, 8, 10, 12, 14, 16, 18, 20, 22, and 24. In calculating percent amino acid sequence identity, two sequences are aligned optimally and the number of identical matches of nucleotides or amino acid residues between the two sequences is determined. The number of identical matches is divided by the length of the aligned region (i.e., the number of aligned amino acid residues) and multiplied by 100 to arrive at a percent sequence identity value. A gap, i.e. a position in an alignment where a residue is present in one sequence but not in the other is regarded as a position with non-identical residues. Amino acid sequence identities may be determined using BLASTX 2.2.14 using the standard settings. The standard settings allow, for example, for sequence gaps in alignments.

The length of the aligned region is preferably over the entire length of the respective protein of SEQ ID NO: 2, 4, 6, 8, 10, 12, 14, 16, 18, 20, 22, or 24.

In another embodiment, the protein to be expressed comprises or consists of an amino acid sequence of at least 80%, preferably at least 90%, more preferably at least 95% of the number of amino acid residues of any one of SEQ ID NO: 2, 4, 6, 8, 10, 12, 14, 16, 18, 20, 22, and 24, and a sequence identity of at least 90% to SEQ ID NO: 2, 4, 6, 8, 10, 12, 14, 16, 18, 20, 22, or 24, respectively, over such length. In another embodiment, the protein to be expressed comprises or consists of an amino acid sequence of at least 80%, preferably at least 90%, more preferably at least 95% of the number of amino acid residues of any one of SEQ ID NO: 2, 4, 6, 8, 10, 12, 14, 16, 18, 20, 22, and 24, and a sequence identity of at least 95% to SEQ ID NO: 2, 4, 6, 8, 10, 12, 14, 16, 18, 20, 22, or 24, respectively, over such length. In these embodiments, the variant protein is preferably still capable of providing the plants with increased abiotic stress resistance of the respective protein of SEQ ID NO: 2, 4, 6, 8, 10, 12, 14, 16, 18, 20, 22, or 24, respectively.

Alternatively, the variant protein may have, or consist of, an amino acid sequence having from 1 to several amino acid additions, substitutions or deletions compared to the amino acid sequence of any one of SEQ ID NO: 2, 4, 6, 8, 10, 12, 14, 16, 18, 20, 22, and 24, wherein the variant protein is preferably still capable of providing said plants with increased abiotic stress resistance. The maximum number of amino acid additions, substitutions or deletions may be at most 20, preferably at most 15, more preferably at most 10, and even more preferably at most 5, whereby the total number of additions, substitutions and additions together determine the number of “amino acid additions, substitutions or deletions”.

In other embodiments, the protein to be expressed has an amino acid sequence similarity of at least 90% compared to the amino acid sequence of any one of SEQ ID NO: 2, 4, 6, 8, 10, 12, 14, 16, 18, 20, 22, and 24 over the entire length of these SEQ ID NOs. Preferably, the sequence identity is at least 95%, more preferably at least 98% over the entire length of these SEQ ID NOs. In another embodiment, the protein to be expressed comprises or consists of an amino acid sequence of at least 80%, preferably at least 90%, more preferably at least 95% of the number of amino acid residues of any one of SEQ ID NO: 2, 4, 6, 8, 10, 12, 14, 16, 18, 20, 22, and 24, and a sequence similarity of at least 90%, preferably at least 95%, more preferably at least 98% to any one of SEQ ID NO: 2, 4, 6, 8, 10, 12, 14, 16, 18, 20, 22, and 24, respectively, over such length.

Sequence similarity of protein sequences uses a substitution matrix with scores for all possible exchanges of one amino acid with another. The degree of similarity between protein sequences can, for example, be evaluated with the BLOSUM 62 substitution matrix (Henikoff and Henikoff, 1992, Proc. Natl. Acad. Sci. U.S.A.89:10915).

The protein capable of providing said plants with increased drought stress resistance may have a cold shock domain, preferably said protein is an RNA chaperone having a binding site for single-stranded RNA. RNA chaperones such as cold shock proteins are believed to resolve misfolded RNA molecules by destabilizing RNA duplexes or binding to single-stranded nucleic acids (see e.g. Hunger et al., J. Bacteriol. 188 (2006) 240-248). Preferably, the protein is or is a variant (as defined above) of B. subtilis CspB of SEQ ID NO: 2. In one embodiment, the protein comprises the amino acid sequence of SEQ ID NO: 2.

Where the protein is a variant of the protein defined by the amino acid sequence of SEQ ID NO:2, said protein has at least the amino acid residues of CspA from E. coli W11, F18, F20, F31, H33, and F34 at positions corresponding to these positions in CspA from E. coli. Positions corresponding to positions in CspA from E. coli can be determined by alignment and correspond to an optimal alignment of the protein sequences.

In one embodiment, the abiotic stress tolerance is drought tolerance, and said protein is plant transcription factor LAS of SEQ ID NO: 8 or GmRE222 of SEQ ID NO: 10 or a variant thereof as defined above. In such embodiment, the plant may be a dicot plant, preferably a Solanaceae plant, and more preferably a tomato plant.

### EXAMPLES

### Reference Example 1

### Determination of Agrobacterium Cell Concentration in Liquid Culture in Terms of Colony Forming Units (cfu)

The concentration of Agrobacterium cells in liquid suspension in terms of colony forming units per ml (cfu/ml) of liquid suspensions can be determined using the following protocol. Cells of Agrobacterium tumefaciens strain ICF 320 transformed with construct pNMD620 were grown in 7.5 ml of liquid LBS medium containing 25 mg/L kanamycin (AppliChem, A1493) and 50 mg/L rifampicin (Carl Roth, 4163.2). The bacterial culture was incubated at 28° C. with continuous shaking. Absorbance or optical density of bacterial culture expressed in absorbance units (AU) was monitored in 1-ml aliquots of the culture using a spectrophotometer at 600 nm wavelength (0D600). The cell concentration estimated as a number of colony-forming units per milliliter of liquid culture (cfu/ml) can be analyzed at OD600 values 1; 1.3; 1.5; 1.7 and 1.8. For this purpose 250-μl aliquots of liquid culture were diluted with LBS-medium to achieve a final volume of 25 ml (dilution 1:100). 2.5 ml of such 1:100 dilution were mixed with 22.5 ml of LBS to achieve the dilution 1:1000. Liquid culture dilutions 1:100; 1:1,000; 1:10,000; 1:100,000; 1:1,000,000; 1:10,000,000 and 1:100,000,000 were prepared similarly. Aliquots of last three dilutions were spread on agar-solidified LBS medium supplemented with 25 mg/L kanamycin and 50 mg/L rifampicin (250 μl of bacterial culture per plate of 90 mm diameter). Plating of aliquots for each dilution was performed in triplicate. After 2 days incubation at 28° C., bacterial colonies were counted for each plate. Plating of 1: 1,000,000 and 1:10,000,000 dilutions resulted in few hundred and few dozen colonies per plate, respectively. So far as dilution 1:100,000,000 provided just few colonies per plate, this dilution was not used for calculation of cell concentration. The cell concentration was estimated according to the formula: cfu/ml =4×number of colonies per plate×dilution factor.

For transforming cell concentrations as measured by absorbance measurements at 600 nm (in LB medium) and in terms of cell-forming units, the following relation is used herein: an OD600 of 1.0 corresponds to 1.1×10⁹ cfu/ml.

### LBS Medium (Liquid)

1% soya peptone (papaic hydrolysate of soybean meal; Duchefa, S1330)

0.5% yeast extract (Duchefa, Y1333)

1% sodium chloride (Carl Roth, 9265.2)

dissolved in water, and the is adjusted to pH 7.5 with 1M NaOH (Carl Roth, 6771.2)

To prepare the solid LBS medium, liquid LBS medium was supplemented with 1.5% agar (Carl Roth, 2266.2). Media were autoclaved at 121° C. for 20 min.

### Example 1

### Vectors Used in the Following Examples

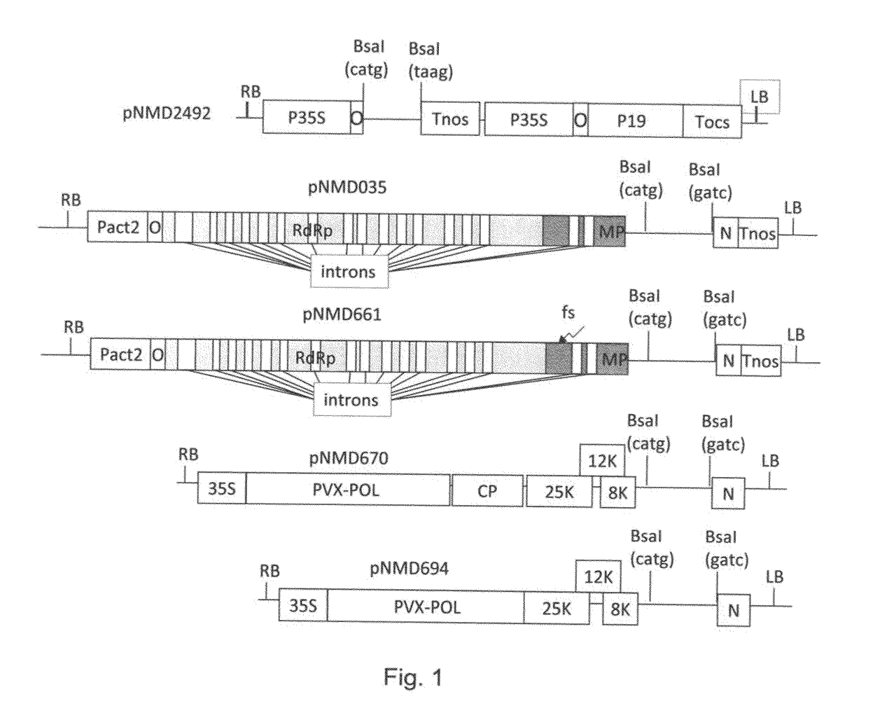

In this study we used standard transcriptional vectors based on 35S CaMV promoter as well as TMV- and PVX-based viral replicons with or without cell-to-cell movement ability. Plasmid maps for all five types of cloning vectors are represented FIG. 1. In all cases the gene of interest is inserted using Bsal cloning sites with catg and gatc (taag) 5′ and 3′ overhangs, respectively. pNMD2492 transcriptional vector was created on the basis of pICBV10, a pBIN19-derived binary vector (Marillonnet et al., 2004, 2006). It contains two expression cassettes inserted between T-DNA right and left borders. An expression cassette adjacent to the right border comprised CAMV 35S promoter, omega translational enhancer, Bsal cloning site for the insertion of gene of interest and the nos terminator (listed in sequential order). Expression cassette adjacent to the left border contained 35S promoter followed by omega translational enhancer, coding sequence of P19 suppressor of silencing from Tomato Bushy Stunt Virus (TBSV) (Gen Bank accession no. CAB56483.1) and ocs terminator.

TMV-based vector with cell-to cell movement ability pNMD035, TMV-based vector lacking cell-to cell movement ability pNMD661, PVX-based vector with cell-to-cell and systemic movement ability pNMD670 and PVX-based vector disabled for both systemic and cell-to cell movement pNMD694 have been already described in the patent “Process for transfecting plants”.

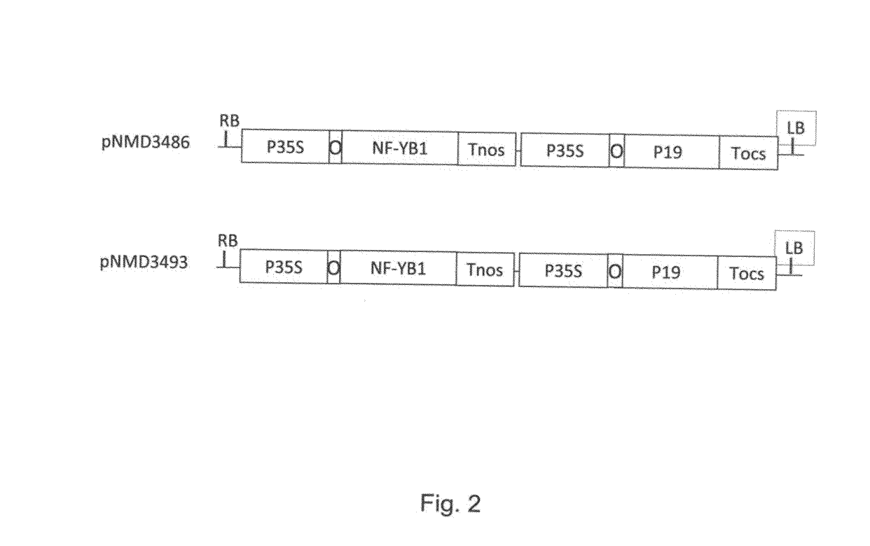

Plasmid construct pNMD3486 was created by direct insertion of coding sequence of Arabidopsis thaliana nuclear transcription factor Y subunit B-1 (NF-YB1) (GenBank: NM_129445.2) into pNMD1426 cloning vector using Bsal cloning site. Similarly, codon-optimized coding sequence of Cold shock protein CspB from Bacillus subtilis subsp. subtilis str JH642 (GenBank: AAB01346) was inserted into same cloning vector resulting in plasmid construct pNMD3493 (FIG. 2).

### Example 2

### Influence of Transient Expression of NF-YB1 and CspB Genes on the Drought Resistance of Rapeseed Plants

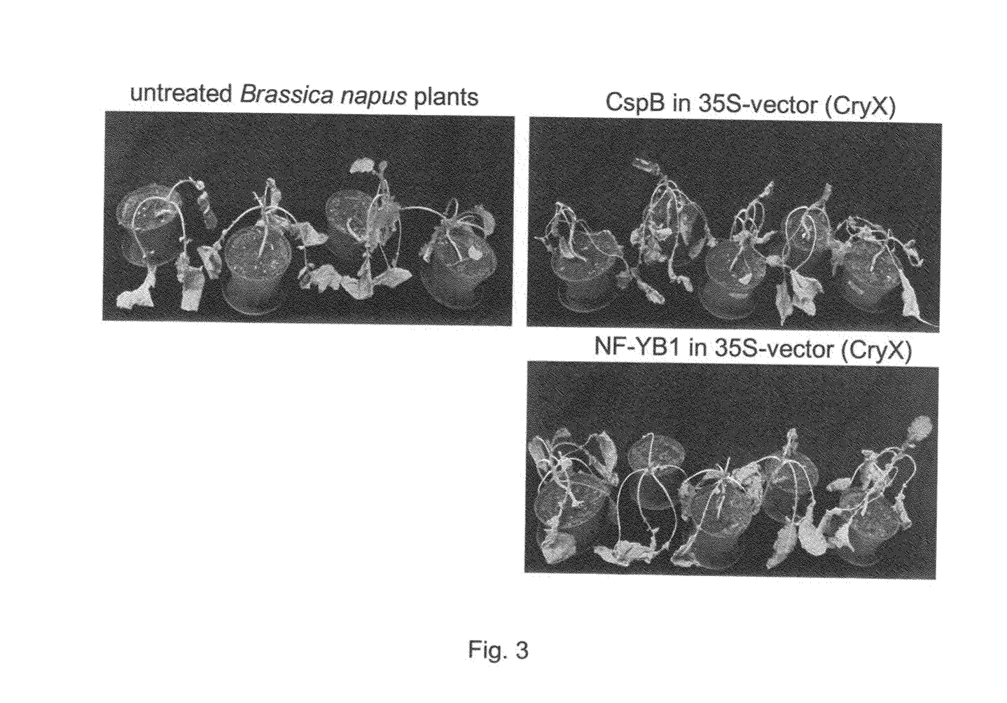

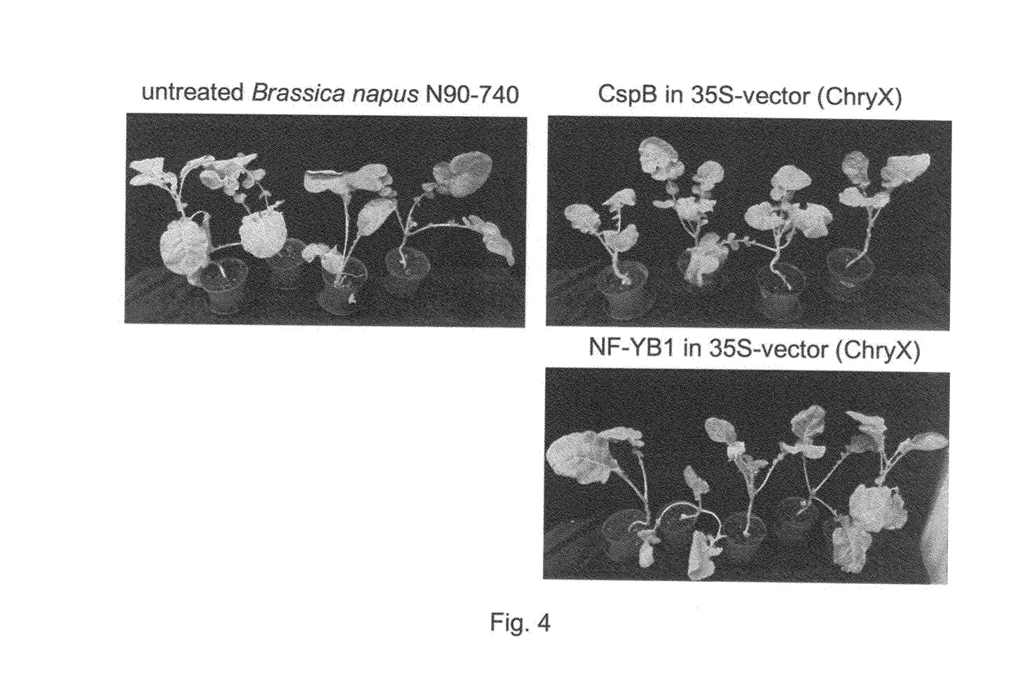

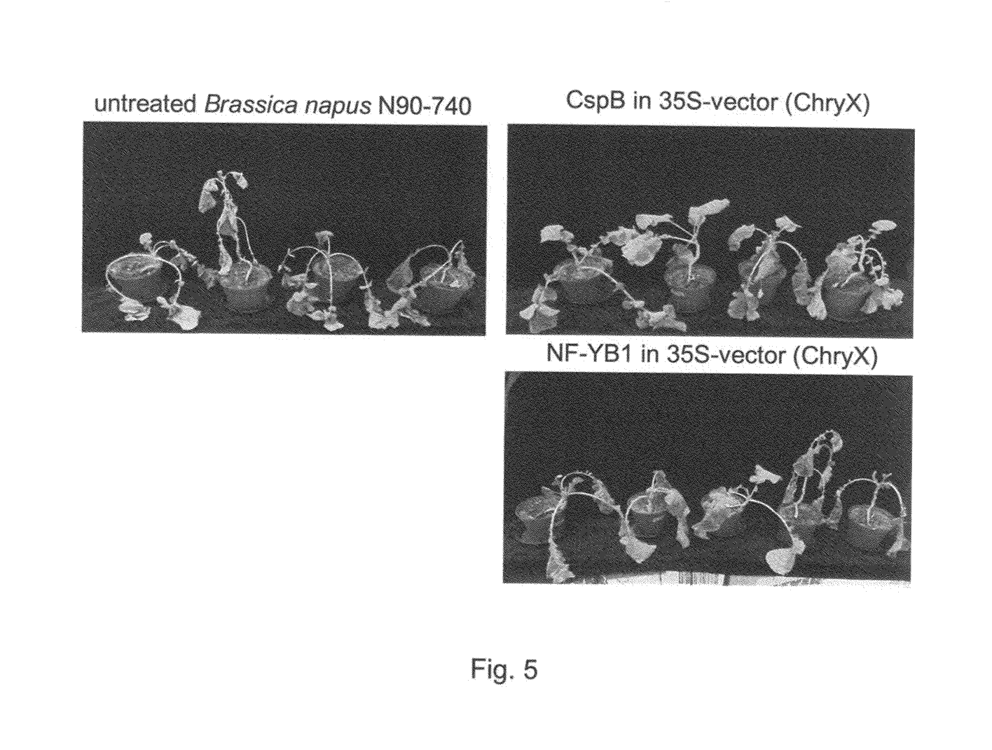

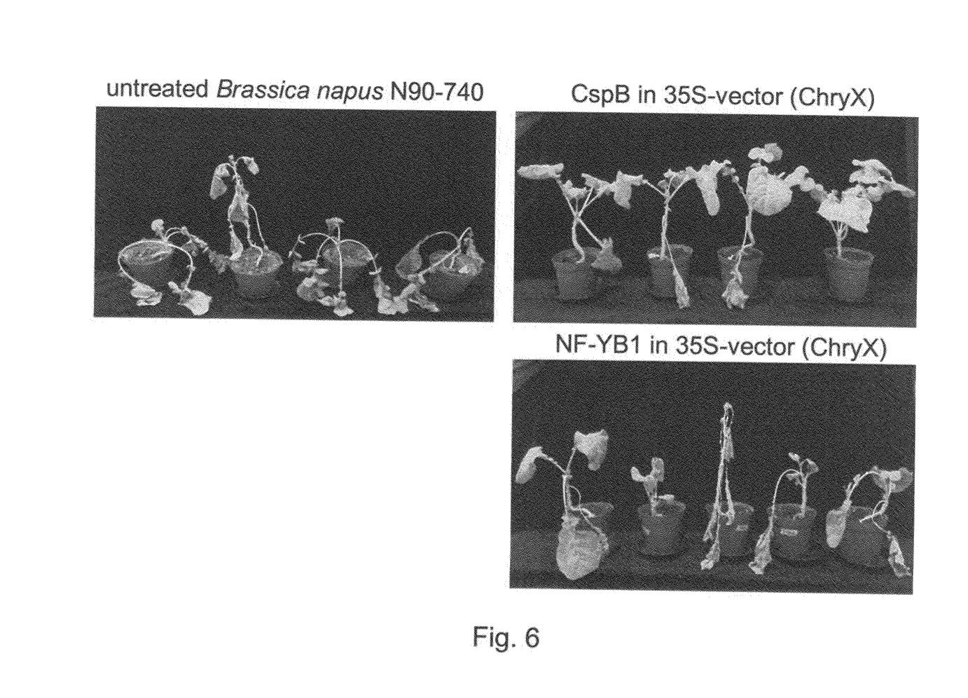

7 weeks old Brassica napus plants were sprayed with Agrobacterium tumefaciens CryX strain harboring pNMD3486 and pNMD3493 constructs (transcriptional vectors containing NF-YB1 and CspB coding sequences under the control of 35S promoter, respectively (FIG. 2). A. tumefaciens strain CryX is described in International Patent Application PCT/EP2013/000994. Watering of plants was stopped on the day of spraying. After 11 days of drought, plants were watered next 7 days. After that plants were subjected to 13 days of drought and watered again for 1 day. By the end of the first drought treatment (11 days) all NF-YB1, CspB and untreated control plants looked wilted without any significant difference between groups (FIG. 3). During next 7 days of watering all plants restored the shape they had before drought treatment, plants looked healthy and vigorous (FIG. 4). By the end the second drought treatment (8 days) CspB plants were slightly less wilted if compared with NF-YB1 plants (FIG. 5). After one day of re-watering CspB plants were much more vigorous than two other groups (FIG. 6).

### Example 3

### Influence of Transient Expression of BnLAS and GmRD22 Genes on the Drought Resistance of Tomato Plants

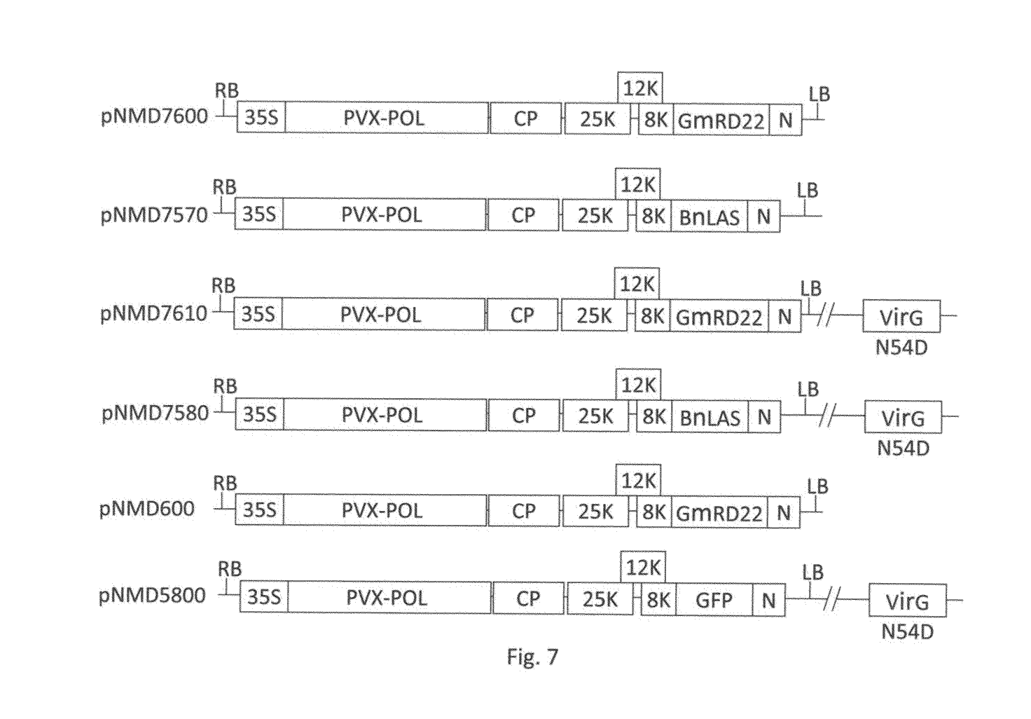

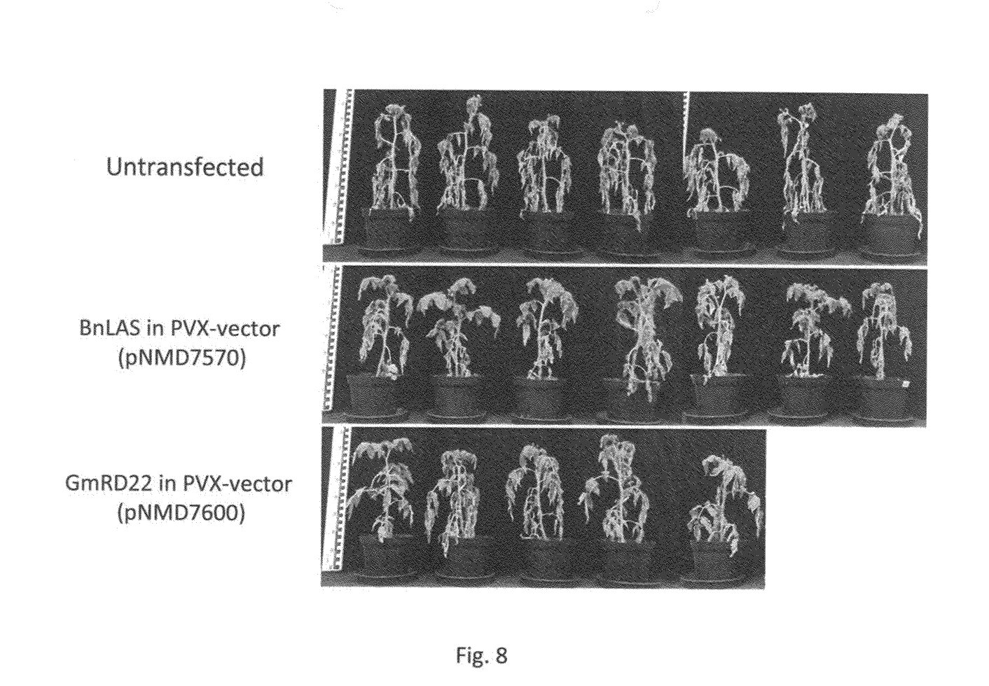

3 weeks old Lycopersicon esculentum cv Balcony Red plants were sprayed with Agrobacterium tumefaciens ICF320 strain harboring pNMD7570, pNMD7600 and pNMD600 constructs (PVX-based vectors with systemic movement ability containing BnLAS, GmRD22 and GFP coding sequences, respectively) (FIG. 7). Watering of plants was stopped 3 weeks post spraying. Drought exposure lasted for 14 days. By the end of drought treatment, BnLAS and GmRD22 transfected plants were less wilted than control untransfected plants (FIG. 8). After 14 days of drought (water saturation of soil approximately 34%), plants were watered again until the fruit harvest.

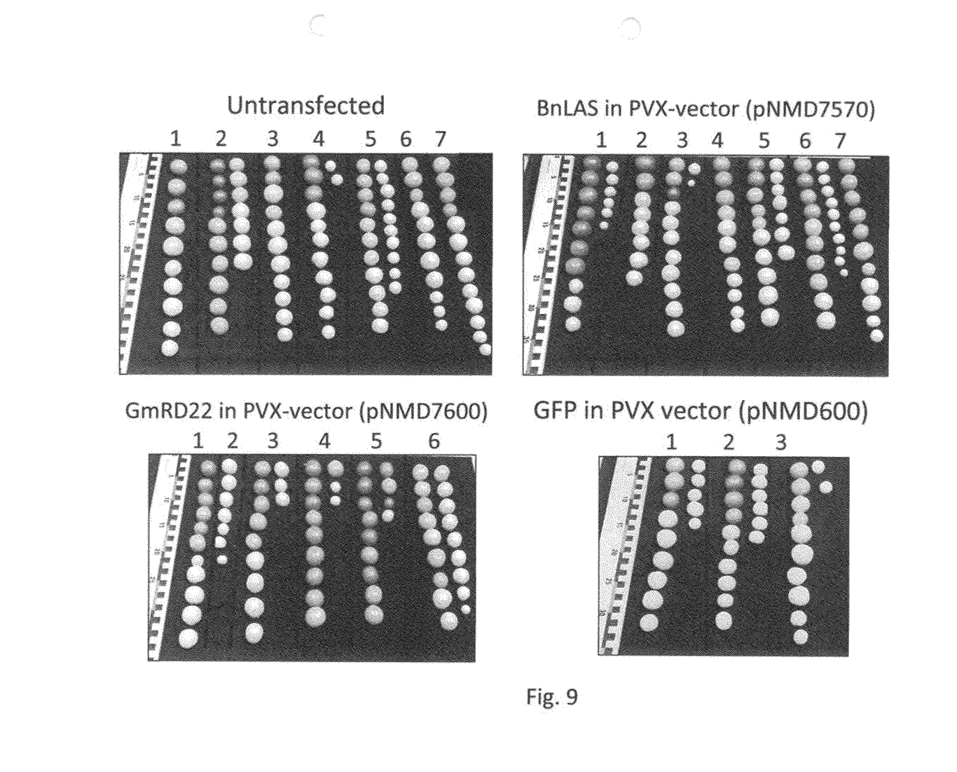

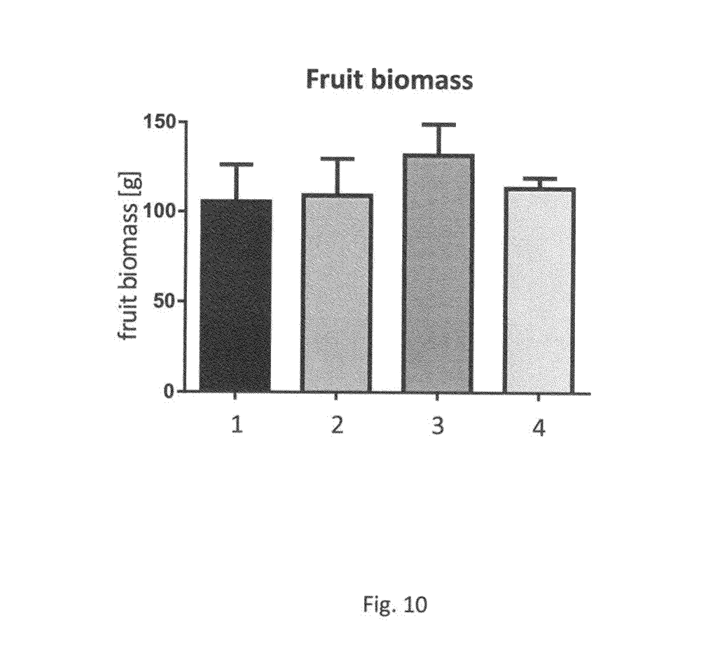

The fruit yield of tomato plants exposed to the drought was evaluated 100 days post spraying with agrobacteria. Fruits harvested for individual plants included in the experiment are shown in FIG. 9. After the drought exposure, GmRD22 transfected plants showed higher fruit biomass if compared with untransfected plants and plants transfected with BnLAS and GFP plants (FIG. 10).

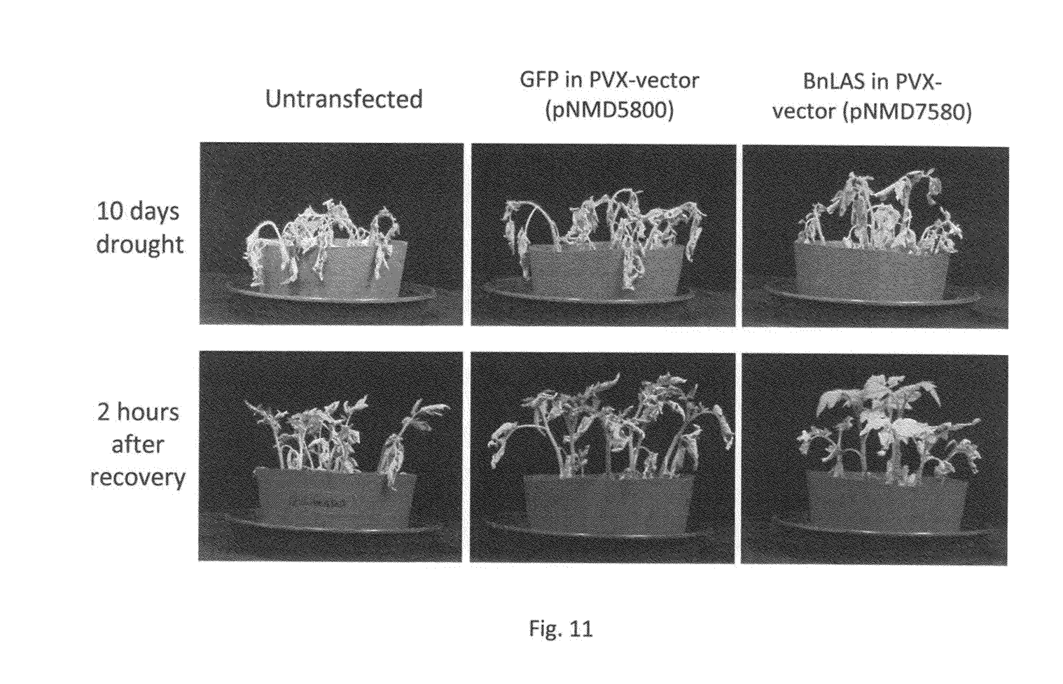

To evaluate the effect of transient expression of BnLAS on the drought resistance of young seedlings, 12 days old tomato (cv Balcony Red) plantlets grown in soil were sprayed with suspension of agrobacteria harboring PVX vectors pNMD7580 (BnLAS insertion) and pNMD5800 (GFP insertion, transfection control). 13 days post spraying, seedlings were exposed to 10 days drought followed by re-watering. After the 10 days drought treatment, BnLAS transfected plants were more vigorous and less wilted compared with untransfected seedlings and seedlings transfected with GFP vectors (FIG. 11, top panel). The difference is even more obvious after 2 hours of water recovery (FIG. 11, bottom panel).

### Example 4

### Influence of Transient Expression of BnLAS and GmRD22 Genes on the Osmotic Stress Resistance of Tomato Plants

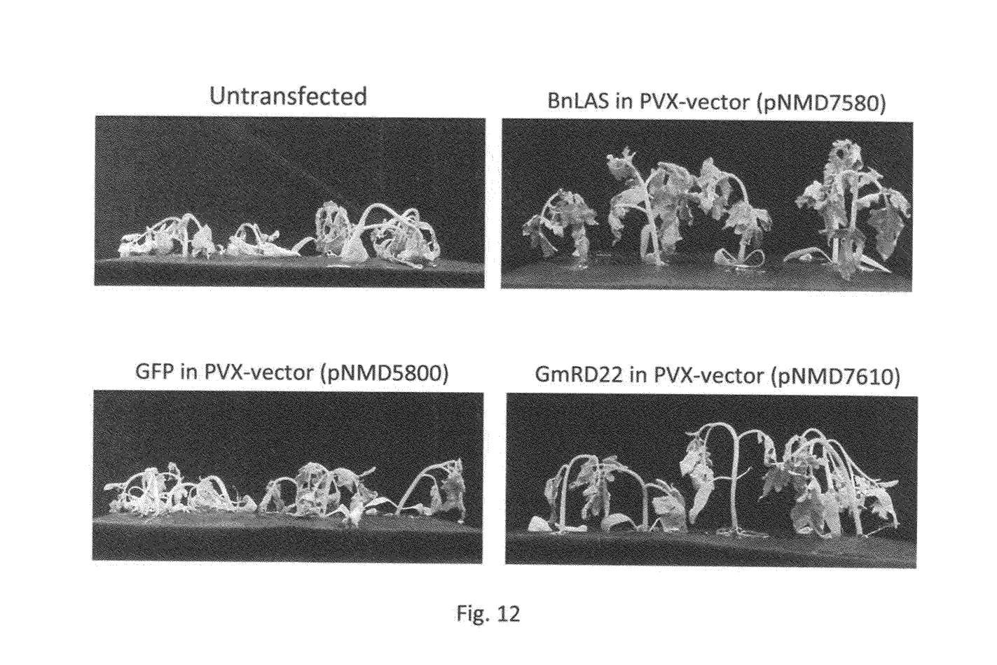

12 days old tomato (cv Balcony Red) seedlings grown in soil were sprayed with suspension of agrobacteria harboring PVX vectors pNMD7580 (BnLAS insertion), pNMD7610 (GmRD22 insertion) and pNMD5800 (GFP insertion, transfection control). 13 days post spraying seedlings were transferred into beakers containing 20% solution of PEG6000 and incubated there for 20 hours. As depicted in FIG. 12, BnLAS and GmRD22 transfected plants were more viable and less wilted when compared with untransfected plants and plants transfected with GFP vectors.

### Nucleic Acid and Amino Acid Sequences Referred to Herein

CspB-cold shock protein from Bacillus subtilis
SEQ ID NO: 1: complete CDS, codon optimized for Brassica napus,
synthesized by Entelechon, GenBank: AAB01346
Atgctagagggcaaagtgaagtggttcaacagcgagaagggattcggctttatcgaagtggaaggccagg
atgacgtgtttgttcacttctctgccatccaaggggaaggattcaagacactggaggaaggacaggcagt
ctcattcgagatagtcgaggggaatagaggacctcaagctgcgaacgttaccaaagaggcttga
SEQ ID NO: 2: Amino acid sequence:
MLEGKVKWFNSEKGFGFIEVEGQDDVFVHFSAIQGEGFKTLEEGQAVSFEIVEGNRGPQAANVTKEA
E. coli cold shock protein 7.4 (cspA) gene, complete cds
GenBank: M30139.1
SEQ ID NO: 3: CDS:
Atgtccggtaaaatgactggtatcgtaaaatggttcaacgctgacaaaggcttcggcttcatcactcctg
acgatggctctaaagatgtgttcgtacacttctctgctatccagaacgatggttacaaatctctggacga
aggtcagaaagtgtccttcaccatcgaaagcggcgctaaaggcccggcagctggtaacgtaaccagcctg
taa
SEQ ID NO: 4: Protein:
Msgkmtgivkwfnadkgfgfitpddgskdvfvhfsaiqndgyksldegqkvsftiesgakgpaagnytsl
NF-YB1-Arabidopsis thaliana nuclear transcription factor Y subunit B-1
SEQ ID NO: 5: ORF original sequence, synthesized by Entelechon
GenBank: NM_129445.2
atggcggatacgccttcgagcccagctggagatggcggagaaagcggcggttccgttagggagcaggatc
gataccttcctatagctaatatcagcaggatcatgaagaaagcgttgcctcctaatggtaagattggaaa
agatgctaaggatacagttcaggaatgcgtctctgagttcatcagcttcatcactagcgaggccagtgat
aagtgtcaaaaagagaaaaggaaaactgtgaatggtgatgatttgttgtgggcaatggcaacattaggat
ttgaggattacctggaacctctaaagatatacctagcgaggtacagggagttggagggtgataataaggg
atcaggaaagagtggagatggatcaaatagagatgctggtggcggtgtttctggtgaagaaatgccgagc
tgg
SEQ ID NO: 6: Amino acid sequence
MADTPSSPAGDGGESGGSVREQDRYLPIANISRIMKKALPPNGKIGKDAKDTVQECVSEF
ISFITSEASDKCQKEKRKTVNGDDLLWAMATLGFEDYLEPLKIYLARYRELEGDNKGSGK
SGDGSNRDAGGGVSGEEMPSW
BnLAS-Brassica napus cultivar Hua shuang No. 5 transcription factor LAS
SEQ ID NO: 7: Complete CDS, synthesized by Eurofins MWG Operon
Splice sites removed: 454-456: AG→TC, C651T
GenBank: HQ324233.1
atgcttacttccttcaaatcctctagctcctcctccgaagatgccaccgagaatcctcctcctcctcctc
cgttatgcctcgcctcatcttctgccgcaacatccgccgctcatcacctccgtcgtctactattcacagc
tgcggatttcatctctcagtccaacgtctccgccgctcaaaacatactctcaatcctctcctcaaactct
tccccttacggggactccacggagcggctcgtccatctcttcaccaaagccttgtccgtacggatcggct
tgtctgaaaacactgccacgtggacagcgaacgaaatggcttctagctccacggtttttacaagcagtgt
atgcaaagaacagttcttgtttcgaaccaagaacaacaacaactctgatctcgagtcttgttactatctt
tggctgaaccaactaacaccgtttattcggttctcccatttaacggcgaaccaagcgatcctcgacgcga
ctgagacaaacaacggtaacggagctttacatatacttgacttagatatatcacaaggacttcaatggcc
tccgttgatgcaagccctagccgagagatcatcatcaaaccctagcagtactccacctccttccctccgc
ataaccggatgtggtcgagatgtaaccgtattaaaccgaaccggagatcggttaacccggtttgctaact
ctctaggtcttcagtttcagtttcacacgcttgtgatcgctgaagaagacctcgccggacttttgcttca
gatcagattattagctctctccgccgtacaaggagagtccatcgccgtcaactgcgtccacttccttcac
agattctttaacgacgacggagacatgatcggtcacttcctgtcggcgatcaagagcttaaaccctagaa
tcgtgacaatggcggagagagaagcgaaccatggagatccttcgttcttgactagattctcagaggcttt
agatcatttcatggcgatatttgattcgttggaagcgactttgccgccaaacagcaaagagaggctaacc
ctagagcaacggtggttcggtatggagattttggatgttgtggcggcggaagcggcggagagaaagcaaa
gacatcggaggtttgaggtttgggaggagatgatgaagagacatggctttgctaacgtgccaataggaag
ctttgctttctctcaagctaagcttctgcttagactccattatccttcagaaggttataatcttcagttt
ctcaacgactctttgtttcttggatggaaaaatcgtcttctcttctccgtttcgtcgtggaaa
SEQ ID NO: 8: Amino acid sequence
MLTSFKSSSSSSEDATENPPPPPPLCLASSSAATSAAHHLRRLLFTAADFISQSNVSAAQNILSILSSNS
SPYGDSTERLVHLFTKALSVRIGLSENTATWTANEMASSSTVFTSSVCKEQFLFRTKNNNNSDLESCYYL
WLNQLTPFIRFSHLTANQAILDATETNNGNGALHILDLDISQGLQWPPLMQALAERSSSNPSSTPPPSLR
ITGCGRDVTVLNRTGDRLTRFANSLGLQFQFHTLVIAEEDLAGLLLQIRLLALSAVQGESIAVNCVHFLH
RFFNDDGDMIGHFLSAIKSLNPRIVTMAEREANHGDPSFLTRFSEALDHFMAIFDSLEATLPPNSKERLT
LEQRWFGMEILDVVAAEAAERKQRHRRFEVWEEMMKRHGFANVPIGSFAFSQAKLLLRLHYPSEGYNLQF
LNDSLFLGWKNRLLFSVSSWK
GmRD22-apoplast-localized BURP-domain protein from soybean
SEQ ID NO: 9: Complete CDS, synthesized by Eurofins MWG Operon (C843G
silent mutation for BsaI site removal)
GenBank: BT097299.1
Atggagtatcgtctcctacccatttttactttactcaatcttgcactggtggcaatccatgctgctttac
ctcctgaagtttactggaagtcggtgcttcctactacgccaatgccaaaagccatcactgatatccttta
ccccgattgggtggaagagaaaagtacctcagtgaatgttggaggcaagggcgtaaacgtgcatgcagga
aaaggaggaggtggcaccaatgtcaacgttggtggaaaaggatcaggcggaggcgtgaacgtgcatgcag
gtcacaagggaaagccagtgcatgtttctgttggctcaaagtctccattcaattacatctacgcttcaac
ggagactcaattacacgatgaccccaacgtcgcactcttcttcttggaaaaggacttgcatcccggaaca
aagttgaacttgcacttcaccaccagttccaatattcaagccacattcttgccacgccaagttgcggatt
ctatacccttttcatccagcaaggtggaggttgtattcaacaagttttccgtaaaacccgggtcagagga
ggcccagatcatgaagaatactctcagtgagtgtgaagagggtggcatcaaaggagaggaaaagtactgt
gccacttcgcttgaatccatgattgatttcagcacttccaagcttggaaaaaatgttgaggttgtgtcca
cggaagtagtggaggacaaggaaacgggattgcagaaatacaccgtagcaccgggagtgaacaagttatc
aggggacaaggctgttgtgtgccacaagcagaactacccttatgctgttttttactgtcacaaaactgag
acgacaagagcttactctgtgcctttggagggtgctaatggggttagggttaaagcggtagcagtgtgcc
acactcacacgtcggaatggaaccctaaacatttggcctttcaagtgctcaaagttaagccaggaaccgt
tcctgtctgccacttcctacctgaggatcatgttgtttgggttcccaag
SEQ ID NO: 10: Amino acid sequence
MEYRLLPIFTLLNLALVAIHAALPPEVYWKSVLPTTPMPKAITDILYPDWVEEKSTSVNVGGKGVNVHAG
KGGGGTNVNVGGKGSGGGVNVHAGHKGKPVHVSVGSKSPFNYIYASTETQLHDDPNVALFFLEKDLHPGT
KLNLHFTTSSNIQATFLPRQVADSIPFSSSKVEVVFNKFSVKPGSEEAQIMKNTLSECEEGGIKGEEKYC
ATSLESMIDFSTSKLGKNVEVVSTEVVEDKETGLQKYTVAPGVNKLSGDKAVVCHKQNYPYAVFYCHKTE
TTRAYSVPLEGANGVRVKAVAVCHTHTSEWNPKHLAFQVLKVKPGTVPVCHFLPEDHVVWVPK
BcWRKY46-Brassica rapa subsp. chinensis transcriptional factor WRKY46
SEQ ID NO: 11: Complete CDS, synthesized by Eurofins MWG Operon.
GenBank: HM585284
Atggctatggaagagaaactcgtgatcaacgaactggaacaagggagagagcttgcccaacgtttgatga
gcaatctcaaagacacttcctcaatcgaatccagcaagaacttgatctctgagatcctcagtatctacca
gaatgctatttccatgttagacgacaagaaggtccttaaacgtagccgtgagatcgatgacaaagattct
aagaacgtgataaaaaagaggcaagtgtttgagaagaagacagagaaagttagtttctttgtcggagcag
gacaagaaaagggttccattgatgatggttattgctggagaaagtacggtcaaaaagagattcatggatc
cattaatccaagaggatatttcagatgcacgcatcgattcacacagaactgtttagcagtgaagcaagtc
caaaaatcagacagagatccttccattttcgaagtgaagtatgtcgggagccacacttgtaacaacacta
ctacgtccccaaagacaccgaacttctctatttcgatgttccaacaagaagacatcaaaccgacgaaaac
agaggaagcgatgatgagtcttgaagatctcgagagcactaagaacattttcagaacgttttctttctcc
aactacgagattgagaatgctggtggtggttggaaaggcaacctcttccatgaggatcagctgtctcctg
ctgcgactacgtcagggtctggaatcaccagcgaggttgcaacagctcctgcttccgttgagaactcgga
gactgcagattcgtatttctcgtctttggacaatattatcgactttggaccggattggttgctgtcgtgt
gacgttttgaattgg
SEQ ID NO: 12: Amino acid sequence
MAMEEKLVINELEQGRELAQRLMSNLKDTSSIESSKNLISEILSIYQNAISMLDDKKVLKRSREIDDKDS
KNVIKKRQVFEKKTEKVSFFVGAGQEKGSIDDGYCWRKYGQKEIHGSINPRGYFRCTHRFTQNCLAVKQV
QKSDRDPSIFEVKYVGSHTCNNTTTSPKTPNFSISMFQQEDIKPTKTEEAMMSLEDLESTKNIFRTFSFS
NYEIENAGGGWKGNLFHEDQLSPAATTSGSGITSEVATAPASVENSETADSYFSSLDNIIDFGPDWLLSC
DVLNW
HaHB1-Helianthus annuus HD-Zip subfamily I trancription factor
SEQ ID NO: 13: Complete CDS, synthesized by Eurofins MWG Operon;
splice sites removed G561A, G750A
GenBank: HQ287802.1
Atgacttgcactggaatggctttcttctcctccaatttcatgttacaatcctcccaagaagatgaccatc
atgcccctacatctctctctccaatcctcccaccttgcagtaccaccactcaagatttcagtggtgctgc
tttcttgggaaaaagatctatgtcttcttactcaggtttgaacaacaacaacatggatggatgtgatcaa
gaagggaacatgaatggagaagatgagttatcagatgatggatcacagcttcttgcaggagagaaaaaga
ggagattaaacatggaacaagtgaagacacttgagagaaactttgagttaggaaataagcttgaacctga
gaggaaaatgcaacttgcaagagcacttggactacaaccaagacagattgctatatggtttcaaaacaga
agagctagatggaaaactaaacagttggaaaaagactatgatgccctcaagagacagtttgaagctgtta
aagctgagaatgattcactccaatctcaaaatcataaacttcatgctgagataatggcactaaaaaatag
agagccagcagaactaatcaacctcaacataaaagaaacagaaggatcttgcagcaaccgaagcgaaaac
agctctgaaatcaaactagacatctcaagaacaccggctaccgatagccctttatcatcacaccatcaac
accaacaccagccaatacctaatctttttccatcgtcgaatatcgatagacctaattcgaataacattgt
ggcgcatcaacttttccacaattcgtcatcaaggccggcagatcatcaacttcattgccacaaactcgat
caatcgaatgccattaaagaagaatgttttagcacaatgtttgttggtatggatgatcaatcagggtttt
ggccatggttggaacaaccacaattcaat
SEQ ID NO: 14: Amino acid sequence
MTCTGMAFFSSNFMLQSSQEDDHHAPTSLSPILPPCSTTTQDFSGAAFLGKRSMSSYSGLNNNNMDGCDQ
EGNMNGEDELSDDGSQLLAGEKKRRLNMEQVKTLERNFELGNKLEPERKMQLARALGLQPRQIAIWFQNR
RARWKTKQLEKDYDALKRQFEAVKAENDSLQSQNHKLHAEIMALKNREPAELINLNIKETEGSCSNRSEN
SSEIKLDISRTPATDSPLSSHHQHQHQPIPNLFPSSNIDRPNSNNIVAHQLFHNSSSRPADHQLHCHKLD
QSNAIKEECFSTMFVGMDDQSGFWPWLEQPQFN
NtAAO-Nicotiana tabacum ascorbate oxidase precursor
SEQ ID NO: 15: Complete CDS, synthesized by Eurofins MWG Operon.
GenBank: D43624
atggcttccttaggcttcttgttcttcttcttgttgccattgattttgcttgagttatcttcatctaggt
cagtaatggcagcaaaaacacggcattttaaatgggacgtggaatatattcattggtcaccagatggtga
agaaagtgtagtaatgggaatcaatggacagtttcctggtccaactattagggcaaaagctggtgatact
gttgctgttcatcttactaacaagctacatactgaaggtgttgtcattcattggcatggaatccgacaga
tcggaacaccatgggctgatggaactgcagcaatttcccaatgcgccattaaccctggagagacatttct
ctataggtttaaagttgataaggcagggacatacttctaccatggacactatggaatgcaaagatcagca
gggctatatggttcactaatagtggaagttggagaaggtgaaaaagaaccattccattatgatggagaat
tcaatttattgcttagtgactggtggcacaaaggttcccatgaacaagaagttgacctctcttccaatcc
tcttcgttggattggtgaaccccagacattgttgctaaatgggagaggtcaatacaattgttcacttgcg
gcgcggtttagcaaaccaccacttccacagtgcaagttaagagggggtgaacagtacgcaccccagattc
tgcgcgtgcgtcccaacaagatttacaggcttagggtggcaagtactactgcattgggttcactcagctt
ggccattgggggtcacaagatggtggtagtagaagcagatggaaactatgttcaaccattttcagtacaa
gacatggacatttattcaggtgaaagctattcagtccttttcaaaacagatcaagatcctaccaaaaact
attggatttcaataaatgtaagaggaagagaaccaaaaacacctcaaggcctcaccttattaaactatct
tccaaattctgcatccaaatttccaactttaccaccacctatagcacccctttggaatgattataaccat
agtaagtcattttctaacaaaatttttgccttaatgggatcacctaagccaccacctcagaaccatcgtc
gtatcatcctgctcaatactcagaacaaaatcgatggttacacgaaatgggctataaataacgtgtcgtt
ggtcttgccaacgcaactttatttaggctcgattagatatggcataaacgcgtttgacacgaaacctcca
ccggacaacttccctaaggactatgatgtcctaaaacaagcaccaaattctaattctacatatggtaatg
gtgtgtatatgctaaagttcaatactacaattgacattatcctacaaaatgcaaatgccttagctaaaga
tgttagtgaaattcatccttggcatttgcatggacatgatttttgggtattgggatatggagaagggaaa
tttagtgaaaaagatgtcaagaagttcaatttaaagaatccaccattgagaaatactgctgtgatttttc
cctttggttggactgcactaagatttgtgacagataatcctggagtttgggcttttcattgtcatattga
gccacatttacatatgggaatgggagttatatttgctgaaggtgttcatcttgtcaagaaaatacctaaa
gaagctttggcttgtggtttgacagggaaaatgttgatgagtaacaagcataat
SEQ ID NO: 16: Amino acid sequence
MASLGFLFFFLLPLILLELSSSRSVMAAKTRHFKWDVEYIHWSPDGEESVVMGINGQFPGPTIRAKAGDT
VAVHLTNKLHTEGVVIHWHGIRQIGTPWADGTAAISQCAINPGETFLYRFKVDKAGTYFYHGHYGMQRSA
GLYGSLIVEVGEGEKEPFHYDGEFNLLLSDWWHKGSHEQEVDLSSNPLRWIGEPQTLLLNGRGQYNCSLA
ARFSKPPLPQCKLRGGEQYAPQILRVRPNKIYRLRVASTTALGSLSLAIGGHKMVVVEADGNYVQPFSVQ
DMDIYSGESYSVLFKTDQDPTKNYWISINVRGREPKTPQGLTLLNYLPNSASKFPTLPPPIAPLWNDYNH
SKSFSNKIFALMGSPKPPPQNHRRIILLNTQNKIDGYTKWAINNVSLVLPTQLYLGSIRYGINAFDTKPP
PDNFPKDYDVLKQAPNSNSTYGNGVYMLKFNTTIDIILQNANALAKDVSEIHPWHLHGHDFWVLGYGEGK
FSEKDVKKFNLKNPPLRNTAVIFPFGWTALRFVTDNPGVWAFHCHIEPHLHMGMGVIFAEGVHLVKKIPK
EALACGLTGKMLMSNKHN
AVP1_ARATH-Arabidopsis thaliana vacuolar H+-pyrophosphatase (AVP-3)
SEQ ID NO: 17: Complete CDS, synthesized by Eurofins MWG Operon;
(with E229D mutation; splices sites removed: A261G, C2004G, A2190T)
GenBank: AEE29349.1
atggtggcgcctgctttgttaccggagctctggacggagatccttgtaccgatttgtgcggtgattggta
tcgccttttcgcttttccaatggtacgttgtatctcgcgtgaaactcacctctgacctcggcgcatcgtc
ttccggtggagctaacaatgggaagaatggatacggtgattatctaatcgaggaagaggaaggtgttaat
gaccagagtgttgtcgctaagtgcgctgagattcagactgctatttccgagggtgcaacttcattcctat
tcacggagtacaaatatgttggtgtcttcatgattttctttgctgctgttatctttgttttcctcggctc
tgttgagggattcagcactgataacaagccttgtacttacgacaccaccagaacctgcaagcctgcattg
gctactgcagctttcagtaccattgctttcgtgcttggtgctgttacctctgttctatctggtttccttg
ggatgaagattgctacatacgctaatgctaggaccactttggaggcgaggaaaggtgttggaaaggcgtt
cattgttgcattcaggtctggtgctgtgatgggtttccttcttgcagcgagtggtctattggtgctttac
attactatcaatgtgttcaagatctattacggagatgactgggaaggtctttttgacgctattactggtt
atggtcttggtgggtcttccatggctctctttggccgtgttggtggtgggatctacactaaggctgctga
tgtcggcgctgaccttgtcggtaaaattgagaggaatattccagaggatgatccaagaaacccagctgtc
attgctgataatgtcggtgacaatgttggtgacattgctggtatgggatctgatctctttggatcatatg
ctgaagcatcatgcgctgctcttgttgttgcctcgatctcatctttcggaatcaaccacgacttcactgc
catgtgctacccattgctcatcagttcaatgggaatcttggtttgtttgatcacaactctctttgccact
gacttctttgagattaagcttgtcaaggagattgaaccagcattgaagaaccagctcattatctcaactg
ttattatgactgttggtattgctattgtgtcatgggttggcttaccgacctcctttaccatcttcaactt
tggaacacaaaaagttgtcaagaactggcagctattcctttgtgtttgtgttggtctttgggctggactc
attattggtttcgtcactgagtactacactagtaacgcctacagccctgtgcaagatgttgcagattcat
gcagaactggtgcagctaccaatgttatcttcggccttgctcttggttacaaatccgtcattattccaat
ctttgctattgctatcagtatattcgttagcttcagctttgctgctatgtatggtgttgctgttgctgct
cttggtatgctcagtaccattgccactggtttggcaattgatgcttatggtcccatcagtgacaatgctg
gtggtattgctgaaatggctggaatgagccaccgcatccgtgaaagaactgatgctcttgatgccgctgg
aaacaccactgctgctattggaaagggatttgccattggctctgctgccctagtctccttggctctcttt
ggtgcctttgtgagccgtgcagggatccacaccgtagatgttttgacccctaaagttatcattgggctcc
ttgttggtgccatgcttccttactggttctctgccatgacaatgaagagtgtgggaagtgcagctcttaa
gatggttgaagaagttcgcaggcagttcaacaccatccctggacttatggaaggaaccgcaaaaccagac
tacgccacatgtgtcaagatctccaccgatgcttccatcaaggaaatgatacctcctggttgccttgtca
tgctcacacctctcattgttggtttcttctttggagttgagacgctctctggtgtcctcgccggatctct
tgtatccggtgttcagatcgccatatcagcatctaacactggtggtgcctgggacaacgccaagaaatac
atcgaggctggtgtatcagagcacgcaaagagccttggaccaaagggttcagagccacacaaggcagctg
tgattggagacacaattggtgacccattgaaggatacttcaggaccttcattgaacatcctcatcaagct
catggctgttgagtctcttgtctttgctcccttcttcgccactcacggtggtatccttttcaagtacttc
SEQ ID NO: 18: Amino acid sequence
MVAPALLPELWTEILVPICAVIGIAFSLFQWYVVSRVKLTSDLGASSSGGANNGKNGYGDYLIEEEEGVN
DQSVVAKCAEIQTAISEGATSFLFTEYKYVGVFMIFFAAVIFVFLGSVEGFSTDNKPCTYDTTRTCKPAL
ATAAFSTIAFVLGAVTSVLSGFLGMKIATYANARTTLEARKGVGKAFIVAFRSGAVMGFLLAASGLLVLY
ITINVFKIYYGDDWEGLFDAITGYGLGGSSMALFGRVGGGIYTKAADVGADLVGKIERNIPEDDPRNPAV
IADNVGDNVGDIAGMGSDLFGSYAEASCAALVVASISSFGINHDFTAMCYPLLISSMGILVCLITTLFAT
DFFEIKLVKEIEPALKNQLIISTVIMTVGIAIVSWVGLPTSFTIFNFGTQKVVKNWQLFLCVCVGLWAGL
IIGFVTEYYTSNAYSPVQDVADSCRTGAATNVIFGLALGYKSVIIPIFAIAISIFVSFSFAAMYGVAVAA
LGMLSTIATGLAIDAYGPISDNAGGIAEMAGMSHRIRERTDALDAAGNTTAAIGKGFAIGSAALVSLALF
GAFVSRAGIHTVDVLTPKVIIGLLVGAMLPYWFSAMTMKSVGSAALKMVEEVRRQFNTIPGLMEGTAKPD
YATCVKISTDASIKEMIPPGCLVMLTPLIVGFFFGVETLSGVLAGSLVSGVQIAISASNTGGAWDNAKKY
IEAGVSEHAKSLGPKGSEPHKAAVIGDTIGDPLKDTSGPSLNILIKLMAVESLVFAPFFATHGGILFKYF
SNAC1-Oryza sativa (japonica cultivar-group) stress-induced Transcription
factor NAC1 (SNAC1)
SEQ ID NO: 19: Complete CDS, codon optimized for Nicotiana
benthamiana, Synthesized by Entelechon
GenBank: DQ394702.1
ATGGGAATGAGACGAGAACGAGACGCAGAAGCCGAGCTTAATCTGCCACCTGGCTTTCGTTTTCATCCGA
CTGATGATGAACTAGTGGAACACTACCTTTGCAGAAAAGCAGCTGGTCAAAGGCTCCCAGTTCCTATCAT
AGCTGAGGTTGATCTCTATAAATTTGACCCCTGGGATCTTCCGGAAAGGGCATTGTTTGGGGCTCGTGAA
TGGTACTTTTTCACCCCACGCGATAGGAAGTACCCAAACGGATCTCGTCCAAACAGGGCCGCTGGAAACG
GTTATTGGAAAGCGACTGGAGCTGACAAGCCAGTAGCTCCTAGGGGGAGAACGCTCGGTATTAAAAAGGC
TCTAGTTTTCTACGCTGGAAAAGCCCCAAGAGGCGTTAAGACTGACTGGATTATGCATGAATATAGACTG
GCTGATGCTGGAAGAGCTGCTGCGGGGGCTAAGAAAGGTAGTTTGAGATTAGACGATTGGGTCCTCTGTC
GGCTATATAACAAGAAGAACGAGTGGGAGAAGATGCAACAGGGGAAAGAGGTTAAGGAGGAAGCCTCGGA
TATGGTGACTTCCCAGTCGCATAGCCACACCCACTCTTGGGGAGAAACCCGCACACCAGAGTCTGAGATC
GTTGATAACGACCCCTTTCCGGAATTGGATTCTTTCCCTGCCTTTCAACCTGCACCTCCACCTGCAACTG
CAATGATGGTCCCGAAGAAAGAATCAATGGACGACGCAACAGCAGCAGCTGCTGCTGCCGCGACAATCCC
TAGGAATAATTCAAGCCTTTTCGTAGATCTTTCCTACGACGATATTCAGGGGATGTATAGCGGCTTAGAC
ATGTTGCCTCCCGGCGACGACTTCTATTCCTCATTGTTCGCAAGTCCCCGTGTCAAAGGAACAACACCTC
GAGCAGGTGCTGGAATGGGAATGGTGCCTTTTTAA
SEQ ID NO: 20: Amino acid sequence
MGMRRERDAEAELNLPPGFRFHPTDDELVEHYLCRKAAGQRLPVPIIAEVDLYKFDPWDL
PERALFGAREWYFFTPRDRKYPNGSRPNRAAGNGYWKATGADKPVAPRGRTLGIKKALVF
YAGKAPRGVKTDWIMHEYRLADAGRAAAGAKKGSLRLDDWVLCRLYNKKNEWEKMQQGKE
VKEEASDMVTSQSHSHTHSWGETRTPESEIVDNDPFPELDSFPAFQPAPPPATAMMVPKK
ESMDDATAAAAAAATIPRNNSSLFVDLSYDDIQGMYSGLDMLPPGDDFYSSLFASPRVKG
TTPRAGAGMGMVPF-
IPT isopentenyl transferase IPT (E88G) from Agrobacterium tumefaciens C58
SEQ ID NO: 21: CDS, E88G Mutation Gen Bank: AB025109.1
Atggatctgcgtctaattttcggtccaacttgcacaggaaagacgtcgaccgcggtagctcttgcccagc
agactgggcttccagtcctttcgctcgatcgggtccaatgttgtcctcagctgtcaaccggaagcggacg
accaacagtggaagaactgaaaggaacgagccgtctataccttgatgatcggcctctggtgaagggtatc
atcgcagccaagcaagctcatgaaaggctgatgggggaggtgtataattatgaggcccacggcgggctta
ttcttgagggaggatctatctcgttgctcaagtgcatggcgcaaagcagttattggagtgcggattttcg
ttggcatattattcgccacgagttagcagacgaggagaccttcatgaacgtggccaaggccagagttaag
cagatgttacgccctgctgcaggcctttctattatccaagagttggttgatctttggaaagagcctcggc
tgaggcccatactgaaagagatcgatggatatcgatatgccatgttgtttgctagccagaaccagatcac
atccgatatgctattgcagcttgacgcagatatggaggataagttgattcatgggatcgctcaggagtat
ctcatccatgcacgccgacaagaacagaaattccctcgagttaacgcagccgcttacgacggattcgaag
gtcatccattcggaatgtat
SEQ ID NO: 22: Amino acid sequence
MDLRLIFGPTCTGKTSTAVALAQQTGLPVLSLDRVQCCPQLSTGSGRPTVEELKGTSRLYLDDRPLVKGI
IAAKQAHERLMGEVYNYEAHGGLILEGGSISLLKCMAQSSYWSADFRWHIIRHELAHEETFMNVAKARVK
QMLRPASGLSIIQELVDLWKEPRLRRILKEIDGYRYAMLFVSQNQITSDMLLQLDADMEDKLIHGIAQEY
LIHARRQEQKFPRVNAAAYDGFEGHPFGMY
LeCYP-Tomato Cyclophilin
SEQ ID NO: 23: CDS
M550019.1; gi: 170439
Atggcaaatccaaaggttttctttgaccttaccatcggtggtgcaccagctggtcgtgtggtgatggagc
tcttcgccgataccactcccaaaaccgctgagaacttccgagctctttgtaccggtgagaaaggtgttgg
aaagatggggaagcctttgcactacaagggctcaaccttccaccgtgtgatcccagggttcatgtgtcaa
ggaggtgatttcaccgccggaaacgggaccggaggagagtcgatctatggagccaaattcaacgatgaga
acttcgttaagaagcacaccggccctggaatcctctccatggctaatgctggacctggaaccaacggttc
tcagtttttcatctgtaccgctaagactgagtggctcaacggaaagcacgtcgtgtttggacaagttgtt
gaaggcatggatgtgattaagaaggcagaggctgttggatctagctctggaaggtgctccaagcctgtgg
ttattgctgactgcggtcaactc
SEQ ID NO: 24: Amino acid sequence
MANPKVFFDLTIGGAPAGRVVMELFADTTPKTAENFRALCTGEKGVGKMGKPLHYKGSTFHRVIPGFMCQ
GGDFTAGNGTGGESIYGAKFNDENFVKKHTGPGILSMANAGPGTNGSQFFICTAKTEWLNGKHVVFGQVV
EGMDVIKKAEAVGSSSGRCSKPVVIADCGQL

SEQ ID NO: 25: T-DNA region of pNMD2492

SEQ ID NO: 26: T-DNA region of pNMD035

SEQ ID NO: 27: T-DNA region of pNMD661

SEQ ID NO: 28: T-DNA region of pNMD670

SEQ ID NO: 29: T-DNA region of pNMD694

SEQ ID NO: 30: T-DNA region of pNMD3486

SEQ ID NO: 31: T-DNA region of pNMD3493

1. A process of providing a plant with improved abiotic stress tolerance, comprising providing aerial parts of said plant with a suspension containing cells of an Agrobacterium strain comprising a nucleic acid molecule comprising a nucleic acid construct containing a nucleotide sequence of interest, said nucleotide sequence of interest providing said plant with increased abiotic stress tolerance upon expression in said plant.

2. The process of providing a plant with improved abiotic stress tolerance according to claim 1, comprising
(i) growing said plant up to a desired growth state; and
(ii) expressing, in said plant, said nucleotide sequence of interest providing said plant with increased abiotic stress tolerance, comprising providing aerial parts of said plant with said suspension containing cells of said Agrobacterium strain.

3. A process of providing a plurality of plants with improved abiotic stress tolerance, comprising
(a) growing said plurality of plants on a farm field;
(b) determining whether an abiotic stress affects said plants;
(c) providing aerial parts of said plants with said suspension containing cells of an Agrobacterium strain comprising a nucleic acid molecule comprising a nucleic acid construct containing a nucleotide sequence of interest, said nucleotide sequence of interest providing said plants with increased abiotic stress resistance upon expression in said plants.

4. The process according to any one of claims 1 to 3, wherein said abiotic stress is drought stress.

5. An agricultural process of producing plants or parts of said plants, comprising the following steps:
(A) growing a plurality of plants on a farm field;
(B) determining whether drought stress affects said plants;
(C) if it was determined in step (B) that drought stress affects or may affect said plants, providing aerial parts of said plants with a suspension containing cells of an Agrobacterium strain comprising a nucleic acid molecule comprising a nucleic acid construct containing a nucleotide sequence of interest, said nucleotide sequence of interest providing said plants with increased drought stress resistance upon expression in said plants;
(D) harvesting said plants or desired parts thereof; and
(E) if step (C) was performed, using the harvest of step (D) for a purpose for which having performed step (C) is acceptable; or, if step (C) was not performed, using the harvest of step (D) for a purpose for which having performed step (C) is not acceptable.

6. The process according to claim 4 or 5, wherein the presence or absence of drought stress affecting said plants is determined using one or more of the following criteria:
water status in the soil such as soil moisture content, water status in tissue of the plants such as relative water content, a plant water stress index, turgor pressure, or stromatal conduct, and water potential in soil or the plant.

7. The process according to any one of claims 1 to 6, wherein said nucleotide sequence of interest encodes a protein capable of providing said plant(s) with said increased abiotic stress tolerance.

8. The process according to claim 7, wherein said protein
i. has an amino acid sequence of any one of the amino acid sequences of SEQ ID NO: 2, 4, 6, 8, 10, 12, 14, 16, 18, 20, 22 and 24; or
ii. has an amino acid sequence having at least 80% sequence identity to the entire amino acid sequence of any one of SEQ ID NO: 2, 4, 6, 8, 10, 12, 14, 16, 18, 20, 22, and 24; or
iii. has an amino acid sequence of a length of at least 80%, preferably at least 90%, more preferably at least 95% of the number of amino acid residues of any one of SEQ ID NO: 2, 4, 6, 8, 10, 12, 14, 16, 18, 20, and 22, and a sequence identity of at least 90% to SEQ ID NO: 2, 4, 6, 8, 10, 12, 14, 16, 18, 20, 22, or 24 respectively, over such length; or
iv. has an amino acid sequence having from 1 to 20 amino acid additions, substitutions or deletions compared to the amino acid sequence of any one of SEQ ID NO: 2, 4, 6, 8, 10, 12, 14, 16, 18, 20, 22, and 24.

9. The process according to any one of claim 7 or 8, wherein said protein is CspB from B. subtilis.

10. The process according to any one of claim 7 or 8, wherein said abiotic stress tolerance is drought tolerance, and said protein is plant transcription factor LAS of SEQ ID NO: 8 or GmRE222 of SEQ ID NO: 10 or a variant thereof as defined in items ii to iv of claim 8.

11. The process according to any one of claims 1 to 10, wherein said plant(s) is (are) dicot, preferably Solanaceae, more preferably tomato plants.

12. The process according to any one of claims 1 to 11, wherein said nucleic acid construct is present in T-DNA and flanked on both sides by T-DNA border sequences, said T-DNA not containing a selectable marker allowing selection of plant or plant cell containing said T-DNA.

13. The process according to any one of claims 1 to 12, wherein said nucleotide sequence of interest is operably linked to a promoter active in plant cells.

14. The process according to any one of claims 1 to 13, wherein said nucleic acid construct encodes a DNA or RNA replicon encoding said protein.

15. DNA molecule or vector for Agrobacterium-mediated transformation of plants, comprising in T-DNA a nucleic acid construct containing a DNA sequence of interest operably linked to a promoter; said DNA sequence of interest encoding a protein capable of providing a plant with increased abiotic stress tolerance; said T-DNA not containing a selectable marker allowing selection of plant cells containing said T-DNA.

16. DNA molecule or vector according to claim 15, wherein said protein is plant transcription factor LAS of SEQ ID NO: 8 or GmRE222 of SEQ ID NO: 10 or a variant thereof as defined in items ii to iv of claim 8.

17. Agrobacterium cell comprising the DNA molecule of claim 15 or 16.

18. Agrobacterium strain comprising a heterologous DNA molecule comprising in T-DNA a nucleic acid construct containing a heterologous DNA sequence of interest operably linked to a promoter; said heterologous DNA sequence of interest encoding a protein capable of providing a plant with increased drought resistance;
said T-DNA not containing a selectable marker allowing selection of plant cells containing said T-DNA.

19. A plant treated with a composition containing cells of an Agrobacterium strain comprising a nucleic acid molecule comprising a nucleic acid construct containing a nucleotide sequence of interest, said nucleotide sequence of interest providing said plant with increased abiotic stress resistance upon expression in said plant; said plant containing in cells thereof said nucleotide sequence of interest and expresses said nucleotide sequence of interest.

20. Method of conferring abiotic stress tolerance to a plant, comprising the following steps: preparing a suspension containing cells of an Agrobacterium strain comprising a nucleic acid molecule comprising a nucleic acid construct containing a nucleotide sequence of interest, said nucleotide sequence of interest providing said plant with increased abiotic stress resistance upon expression in said plant; and providing the suspension prepared in the previous step to aerial parts of said plant.
